(19) United States
(12) Patent Application Publication
(10) Pub. No.: US 2024/0185961 A1
(43) Pub. Date: Jun. 6, 2024

(54) DRUG MATERIAL INTERACTIONS USING QUARTZ CRYSTAL MICROBALANCE SENSORS

(71) Applicant: Janssen Biotech, Inc., Horsham, PA (US)

(72) Inventors: Ligi MATHEWS, Chester Springs, PA (US); Joseph WEIDMAN, Mount Joy, PA (US)

(21) Appl. No.: 18/553,272

(22) PCT Filed: Mar. 31, 2022
(86) PCT No.: PCT/US2022/022711

§ 371 Date: Sep. 29, 2023

Related U.S. Application Data
(60) Provisional application No. 63/177,781, filed on Apr. 21, 2021.
(60) Provisional application No. 63/177,784, filed on Apr. 21, 2021.
(60) Provisional application No. 63/177,786, filed on Apr. 21, 2021.
(60) Provisional application No. 63/169,731, filed on Apr. 1, 2021.
(60) Provisional application No. 63/169,735, filed on Apr. 1, 2021.
(60) Provisional application No. 63/169,737, filed on Apr. 1, 2021.

(51) Int. Cl. G16C 20/30; G16C 60/00

(52) U.S. Cl. CPC G16C 20/30; G16C 60/00

(57) ABSTRACT

Data is received that identifies a medication comprising a concentration of a drug product in a background fluid and a composition of a surface of a receptacle for housing the medication. Thereafter, a drug substance adsorption behavior model executed by at least one computing device is used to predict a percent of dose lost and an interaction behavior between the medication and the receptacle. Thereafter, data is provided that characterizes the predicted percent of dose lost and the interaction behavior. The drug substance adsorption behavior model can be informed using quartz crystal microbalance (QCM) sensors that are exposed to medications and are coated with materials designed to mimic exemplary receptacles. Related apparatus, systems, techniques, and articles are also described.

### CROSS-REFERENCE TO RELATED APPLICATIONS

This application claims the benefit of U.S. Pat. App. Ser. No. 63/169,731 filed Apr. 1, 2021, U.S. Pat. App. Ser. No. 63/169,735 filed Apr. 1, 2021, U.S. Pat. App. Ser. No. 63/169,737 filed Apr. 1, 2021, U.S. Pat. App. Ser. No. 63/177,781 filed Apr. 21, 2021, U.S. Pat. App. Ser. No. 63/177,784 filed Apr. 21, 2021, and U.S. Pat. App. Ser. No. 63/177,786 filed Apr. 21, 2021, the disclosure of each of which is incorporated by reference herein it is entirety.

### TECHNICAL FIELD

The subject matter described herein relates to advanced techniques for characterizing interactions between drugs and materials that utilizes a quartz crystal microbalance sensor.

### BACKGROUND

Food and Drug Administration (FDA) approved investigational monoclonal antibodies (mAbs), and other biologics are used in various formulations and concentrations to treat an ever-growing number of diseases. As formulation development progresses for protein drugs, it is not only important to consider the microbiological stability and a shelf-life, but also the formulation in the final state before it is administered to the patient, and this includes protein aggregation and adsorption to polymer materials of construction. Aggregation and adsorption can affect product quality and can also affect patient safety due to the loss of effective drug substance on materials and in aggregates as well due to formation of immunogenic complexes which could lead to adverse events. Challenges remain to optimize the formulation with each protein and conduct regulatory mandated in use compatibility testing.

### SUMMARY

In a first aspect, data is received that identifies a medication comprising a concentration of a drug product in a background fluid and a composition of a surface of a receptacle for housing the medication. Thereafter, a drug substance adsorption behavior model executed by at least one computing device is used to predict a percent of dose lost and an interaction behavior between the medication and the receptacle. Thereafter, data is provided that characterizes the predicted percent of dose lost and the interaction behavior. The drug substance adsorption behavior model can be informed using quartz crystal microbalance (QCM) sensors that are exposed to medications and are coated with materials designed to mimic exemplary receptacles.

The drug substance adsorption behavior model can be generated by conducting a plurality of test measurements simulating delivery of the medication at various concentrations and with sometimes differing surfactant to protein ratios housed within receptacles having varying sizes and surface compositions. Acoustic resonances of a QCM sensor can be measured during each test measurement. These QCM sensors can have a coating corresponding to the surface composition of the respective receptacle. With this arrangement, different frequencies of measured harmonics forming part of the acoustic resonances correlate to adsorbed drug product by the surface composition. A percent of dose lost and interaction behavior between the medication and receptacle can be determined for each test measurement based on the measured acoustic resonances and arrangement of applicable equations to the model and data based on surfactant to protein ratios in solution. These experimentally determined percent of dose lost measurements and the corresponding interaction behaviors can be used to construct the drug substance adsorption behavior model.

The interaction behavior between the surface of the receptacle and the medication can include how much of a surfactant or other component of the drug solution is adsorbed by the surface of the receptacle.

The predicted percent of dose lost can be based on various factors including a period of time, an amount of dose lost during administration of the medication, an amount of dose lost during manufacture or preparation of the medication, an amount of dose lost during storage of the medication, and/or an amount of dose lost during transportation of the medication.

The received data can include a total possible medication contact surface area for the receptacle.

The receptacle can take various forms including, but not limited to, an intravenous fluid (IV) bag, IV line, a syringe, a pre-filled syringe, an inline filter, a needle, a catheter, intravenous tubing, a vial, or any other surface involved in the manufacture, storage, administration, preparation, or transportation of the drug product.

The surface composition can take various forms including, for example, polyvinyl chloride (PVC), polypropylene (PP), polyvinylidene flouride (PVDF), polyethersulfone (PES), polyethylene (PE), polycarbonate (PC), polyurethane (PUR), nylon, boro-silicate glass, and/or steel. More generally, the surface composition can, for example comprise or be, basic elements, oxides, nitrides, carbides, sulfides, polymers, functionalized molecules, glasses, steels, and/or alloys.

The background fluid can take many forms, including, but not limited to normal saline (NS), half-normal saline, 3% normal saline, lactated Ringer's solution, plasmalyte, dextrose 5% in water, dextrose 5% in water and half-normal saline, dextrose 5% and lactated Ringer's solution, 7.5% sodium bicarbonate, albumin 5%, albumin 25%, 10% dextran 40 in NS, hetastarch 6% in NS, normosol-r, normosol-m., and hypertonic saline.

The providing data characterizing the predicted percent of dose lost and the interaction behavior between the receptacle and the medication can include one or more of: causing the data to be displayed in electronic visual display, transmitting the data over a computing network to a remote computing system, loading the data into memory, or storing the data in physical persistence.

The drug product can take varying forms including a protein, a nucleic acid, a lipid or a virus that is adsorbed by the surface of the receptacle. When the drug product is or includes a protein, the protein can take various forms such as an antibody, an antibody-drug conjugate, or a fusion protein that contacts the surface of the receptacle.

Different modeling approaches can be utilized depending, for example, on the molar ratio of surfactant to protein. These approaches can be selected, for example, based on a shielding point. Shielding point, in this context, can refer to a state at which a protein and surfactant approach a ratio where just above it, the surfactant acts as an adequate shield. When there is low surfactant, the protein approaches too high of a concentration relative to the surfactant to be adequately shielded. When there is high surfactant, the protein approaches too low of a concentration relative to the surfactant to not be adequately shielded.

In some variations (e.g., scenarios in which the molar ratio of surfactant to protein is below a shielding point, etc.), the drug substance adsorbance behavior model can be further generated by estimating a contribution of mass of protein at the surface equal to z (1−x/y). Shielding point, in this context, can refer to a state at which a protein and surfactant approach a ratio where just above it, the surfactant acts as an adequate shield. When there is low surfactant, the protein approaches too high of a concentration relative to the surfactant to be adequately shielded. When there is high surfactant, the protein approaches too low of a concentration relative to the surfactant to not be adequately shielded. In this arrangement, x is a measured adsorbed mass of the medication in a first state, y is a measured adsorbed mass of the medication in a second state, and z is a measured adsorbed mass of the medication in a third state. The drug substance adsorbance behavior model can be further generated by estimating a contribution of mass of protein at the surface equal to z*(x/y).

In other variations (e.g., scenarios in which the molar ratio of surfactant to protein is above a shielding point, etc.), the drug substance adsorbance behavior model can be further generated by estimating a contribution of mass of protein at the surface equal to z (1−y/x). In this arrangement, x is a measured adsorbed mass of the medication in a first state, y is a measured adsorbed mass of the medication in a second state, and z is a measured adsorbed mass of the medication in a third state. The drug substance adsorbance behavior model can be further generated by estimating a contribution of mass of protein at the surface equal to z*(y/x).

In some variations, the shielding point can refer to a molar ratio of 280 surfactant to protein such that molar ratios of 3-280 surfactant to protein are deemed to be below the shielding point and molar ratios of 281-2820 surfactant to protein are deemed to be above the shielding point.

In an interrelated aspect, polymers for medication receptacles can be screened by receiving data identifying a medication comprising a concentration of a drug product in a background fluid and a polymeric composition of a surface of a receptacle for housing the medication. Thereafter, a drug substance adsorption behavior model by at least one computing device predicts a percent of dose lost and an interaction behavior between the medication and the receptacle using the received data. The drug substance absorption behavior model can be generated using one or more empirical tests using quartz crystal microbalance sensors. Thereafter, data is provided that characterizes the predicted percent of dose lost and the interaction behavior.

The predicated percent of dose lost and the interaction behavior can be used to fill or otherwise load a receptacle with the medication. Various factors can be taken into account when selecting the type of receptable for a particular medication such as microbiological stability, shelf-life and the final state of the medication before it is administered to the patient.

The subject matter described herein provides many advantages. For example, the current subject matter can help ensure that medications continue to have their desired pharmacological effect and dosing strength after interacting with various, potentially adsorbing surfaces. Proteins and other large molecular entities must largely retain an active conformation of their structure in the face of interfacial stressors to have their pharmacological effect, and this structure may be lost before, during, or after adsorption to solid surfaces, leading to possible drug loss and aggregation if not reversible or mitigated.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a diagram illustrating drug substance adsorption behavior models based on surfactant concentration relative to protein concentration;

FIG. 2 is a process flow diagram illustrating the characterization of medication and surface interactions using a quartz crystal microbalance;

FIG. 3 is an architecture diagram of aspects of a quartz crystal microbalance instrument;

FIG. 4 is a diagram illustrating top and bottom views of a quartz crystal microbalance (QCM) sensor;

FIG. 5 is a diagram illustrating an experimental run of a QCM to determine mass adsorbed at a sensor surface;

FIG. 6 is a diagram illustrating estimates of mass contributions of surfactant and protein to a layer at two different polymer sensor surfaces at different concentrations;

FIG. 7 is a diagram illustrating measurements of adsorbed masses of only protein, only surfactant, and protein and surfactant in formulated solution diluted in diluent;

FIG. 8 is a diagram illustrating concentration of protein in solution versus estimates of mass contributions of protein to adsorbed layer at two different polymers sensor surfaces;

FIG. 9 is a diagram illustrating electrochemiluminescence immunoassay (ECLIA)-measured percent of dose lost on an IV Set versus QCM estimated mass left on the IV set;

FIG. 10 is a diagram illustrating ECLIA-estimated mass left on a polymer IV set vs QCM estimated mass left on the polymer IV set;

FIG. 11 is a diagram illustrating measurements of adsorbed masses of only protein, only surfactant, and protein and surfactant in a formulated solution diluted in a diluent to a polymeric surface often found in syringes used for subcutaneous administration;

FIG. 12 is a diagram illustrating estimates of mass contributions of surfactant and a protein to a polymeric surface often found in syringes used for subcutaneous administration at different sensor surfaces at different concentrations;

FIG. 13 is a first diagram illustrating a relationship between concentration and adsorbed protein mass;

FIG. 14 is a second diagram illustrating a relationship between concentration and adsorbed protein mass;

FIG. 15 is a diagram illustrating QCM estimated amount adsorbed of a protein versus percent of dose not given by content assay for different surfaces assuming no more than 100% recovery;

FIG. 16 is a diagram illustrating QCM estimated amount adsorbed of a protein versus percent of dose not given by content assay for different surfaces;

FIG. 17 is a diagram illustrating QCM estimated amount adsorbed of a protein per area versus mass of dose per area not given by content assay for different surfaces;

FIG. 18 is a diagram illustrating estimates of mass contributions of surfactant and a protein to layer at different sensor surfaces at different concentrations;

FIG. 19 is a diagram illustrating measurements of adsorbed masses of only protein, only surfactant, and protein and surfactant in formulated solution diluted in diluent;

FIG. 20 is a diagram illustrating a relation of mass contributions of surfactant and a protein to layer at different sensor surfaces at different concentrations;

FIGS. 21A-D are diagrams illustrating filtered and unfiltered models of percent recovery and QCM results;

FIG. 22 is a diagram illustrating estimates of mass contributions of surfactant and a protein to layer at different sensor surfaces at different concentrations;

FIG. 23 is a diagram illustrating measurements of adsorbed masses of only protein, only surfactant, and protein and surfactant in formulated solution diluted in diluent;

FIG. 24 is a diagram illustrating estimates of protein average contributions of the protein part of the layer at the sensor surface at different surfactant concentrations;

FIGS. 25A-D are diagrams illustrating filtered models and unfiltered models of percent recovery and QCM results; and

FIG. 26 is a diagram illustrating estimates of mass contributions of surfactant and a protein to layer at different sensor surfaces at different concentrations.

### DETAILED DESCRIPTION

The current subject matter is directed to enhanced techniques for characterizing dosage losses and interaction behavior between medication and a receptacle surface using a drug substance adsorption behavior model. In particular, the current subject matter is directed to the use of a quartz crystal microbalance (QCM) instrument with dissipation monitoring (sometimes referred to as QCM-D) to generate a drug substance adsorption behavior model which is utilized in one or more computer-implemented algorithms that characterize the interaction of a medication with various materials. These materials form surfaces on various receptacles (e.g. intravenous fluid (IV) bags, IV lines, syringes including pre-filled syringes, inline filters, needles, catheters, tubing sets, vials, etc.) throughout the lifecycle of the medication from initial manufacture, to transportation, and ultimately to preparation and administration to a patient. Medication as used herein includes different biologic drugs, formulations, large or large molecule biologic therapeutics, and materials, or any other molecular or otherwise entity with the intent for use as a drug.

QCM-D comprises an acoustic sensor, which is a resonating piezoelectric A-T cut quartz crystal where resonance is measured at different harmonics of the base resonance frequency and changes in mass and thickness of adlayers at the surface of the acoustic sensor which is exposed to a drug solution can be found. QCM-D can accurately predict the mass as well as viscoelasticity and other properties of the adsorbed layer with mass being used herein to indicate how much drug is lost to adsorption. In other words, the sensor (or sensors) forming part of the QCM-D instrument can have coatings that mimic a medicine receptacle that is to be characterized or otherwise modeled. The Sauerbrey equation holds true when dealing with the masses, adlayers, and proteins in the formulation using QCM. The Sauerbrey equation (equation 1 below) relates the change in the resonance frequency proportionally to the change in the total adsorbed sensor surface mass where ρq and μq are the density (2.648 g·cm−3) and shear modulus of quartz (2.947×1011 g·cm−1·s2), respectively, A is the crystal piezoelectrically active geometrical area, defined by the area of the deposited film on the crystal, f₀ is the unloaded crystal frequency, and Δm and Δf are the mass and system frequency changes.

$$Δ f = - \frac{2 {f}_{0}^{2}}{A \sqrt{{ρ}_{q} {μ}_{q}}} Δ m \qquad Eq . ( 1 )$$

$$Δ f = - {f}_{0}^{( \frac{3}{2} )} \sqrt{\frac{{ρ}_{L} η}{π {ρ}_{q} {μ}_{q}}} \qquad Eq . ( 2 )$$

The derived Kanazawa-Gordon equation (equation 2 above), where f₀ is the unloaded crystal frequency, μ_{q} is shear modulus of quartz, ρ_{q} is the density of quartz, η and ρ_{L} are the liquid viscosity and the density, respectively, deals with when one side of the quartz crystal is immersed in liquid and accounts for the liquid's viscous damping effects while mass is adsorbing, and measurement takes place. Both equations can be used to predict adsorption of mass to the surface of the sensor in a flowing liquid.

The assumptions, which are met in the current method, in order for these relationships to exist and produce meaningful data are that the adsorbed mass must be small relative to the mass of the quartz crystal, the mass adsorbed is a rigid, non-slipping film, and the mass adsorbed is evenly distributed over the area of the crystal.

Different adsorbance modeling approaches can be utilized depending, for example, on the molar ratio of surfactant to protein. These approaches can be selected, for example, based on a shielding point. Shielding point, in this context, can refer to a state at which a protein and surfactant approach a ratio where just above it, the surfactant acts as an adequate shield. When there is low surfactant, the protein approaches too high of a concentration relative to the surfactant to be adequately shielded. When there is high surfactant, the protein approaches too low of a concentration relative to the surfactant to not be adequately shielded.

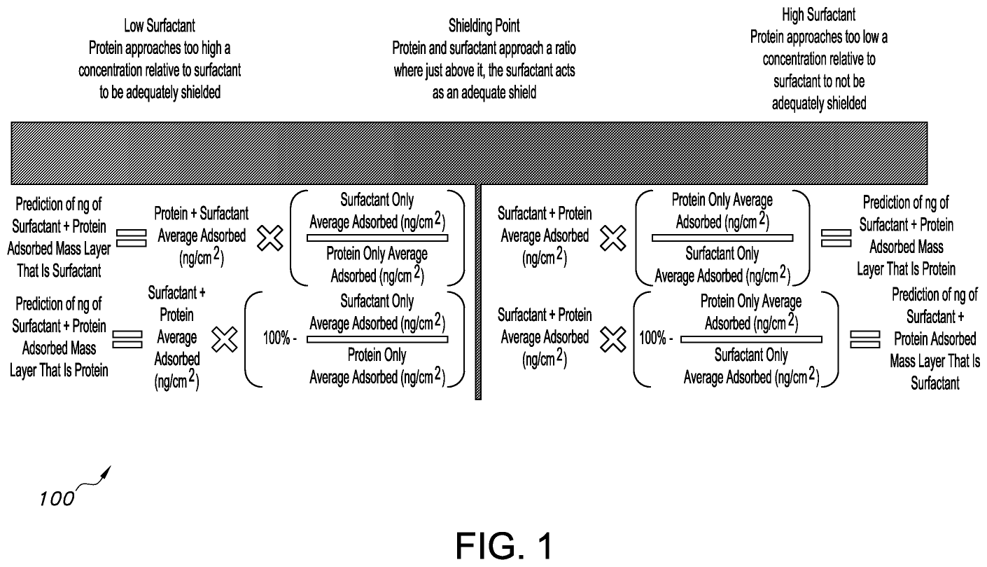

With reference to diagram 100 of FIG. 1, it was found that mass contribution calculations of proteins and surfactant can vary based on the level of surfactant relative to a shielding point. With lower surfactant levels, the mass contribution estimate of protein can be governed by equation 3 and the mass contribution estimate of surfactant can be governed by equation 4. Such a state can occur when the protein approaches too high of a concentration relative to the concentration of surfactant to be adequately shielded from the polymer surface

$$Mass Contribution Estimate of Protein at Material Surface = z ( 1 - \frac{x}{y} ) \qquad ( Eq . 3 )$$

$$Mass Contribution Estimate of PS 20 at Material Surface = z \times \frac{x}{y} . \qquad ( Eq . 4 )$$

Here, variables x, y, and z can be arranged depending on solution characteristics and observance of surfactant to protein ratio to estimate a contribution of mass of protein at the surface, and all represent different characteristic adsorption of drug or other substances in solution that adsorb to the surface.

When there are higher surfactant levels in which there is a high surfactant concentration (i.e., surfactant level is above an estimated shielding point, equation 5 below can apply to calculate the mass contribution estimate of the surfactant at the surface and equation 6 below can be used to calculate mass contribution estimate of the protein at the material surface. With this state, the protein approaches too low of a concentration relative to the polymer surface (e.g., PS, etc.) to be adequately shielded. There can also be a shielding point which corresponds to when the protein and surfactant approach a ratio at which, above such ratio, the surfactant acts as a shield.

$$Mass Contribution Estimate of PS 20 at Material Surface = z ( 1 - \frac{y}{x} ) \qquad ( Eq . 5 )$$

$$Mass Contribution Estimate of Protein at Material Surface = z \times \frac{y}{x} \qquad ( Eq . 6 )$$

With equations 4-6, x is a measured adsorbed mass of the medication in a first state, y is a measured adsorbed mass of the medication in a second state, and z is a measured adsorbed mass of the medication in a third state.

In some variations, the shielding point can refer to a molar ratio of 280 surfactant to protein such that molar ratios of 3-280 surfactant to protein are deemed to be below the shielding point and molar ratios of 281-2820 surfactant to protein are deemed to be above the shielding point.

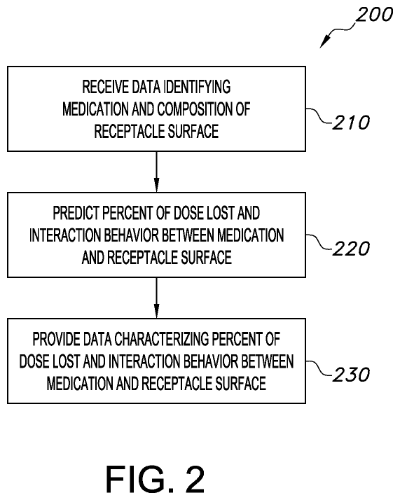

FIG. 2 is a process flow diagram 200 of a computer-implemented process in which, at 210, data is received that identifies a medication comprising a concentration of a drug product in a background fluid and a composition of the material of a surface of a receptacle for housing the medication. Thereafter, at 220, a percent of dose lost and an interaction behavior between the medication and the receptacle surface is predicted by a drug substance adsorption behavior model using the received data. Subsequently, at 230, data is provided (e.g., displayed, transmitted to a remote computing device, loaded into memory, stored in physical persistence, etc.) which characterizes the predicted percent of dose lost and the interaction behavior. Various drug products can be characterized including, cell-based therapeutics, protein therapeutics, viral therapeutics, DNA therapeutics, IgG proteins, and the like. In some cases, the drug product includes one or more of a protein, a nucleic acid, a lipid or a virus that is adsorbed by the surface of the receptacle. When the drug product is or includes a protein, the protein can take various forms such as an antibody, an antibody-drug conjugate, or a fusion protein that contacts the surface of the receptacle.

The drug substance adsorption behavior model can be generated by conducting a plurality of test measurements simulating delivery of the medication at various concentrations housed within receptacles having varying sizes and surface compositions. During each test measurement, acoustic resonances of a QCM sensor having a coating corresponding to the surface composition of the respective receptacle are measured. With such sensors, different frequencies of measured harmonics forming part of the acoustic resonances are directly related to the mass of an adsorbed substance when drug product is exposed to the sensor surface. Both percent of dose lost and interaction behavior between the medication and receptacle material can be subsequently determined for each test measurement based on the measured acoustic resonances. The drug substance adsorption behavior model can be constructed based on the determined percent of dose lost and the interaction behavior and/or measured adsorbed masses measured by QCM between the respective medications and the corresponding receptacles. A medical receptacle suitable for a particular medication can be filled with such medication based on the determined percent of dose lost and the interaction behavior and/or measured adsorbed masses measured by QCM between the respective medications and the corresponding receptacles. Various factors can be taken into account when selecting the type of receptable for a particular medication such as microbiological stability, shelf-life and the final state of the medication before it is administered to the patient.

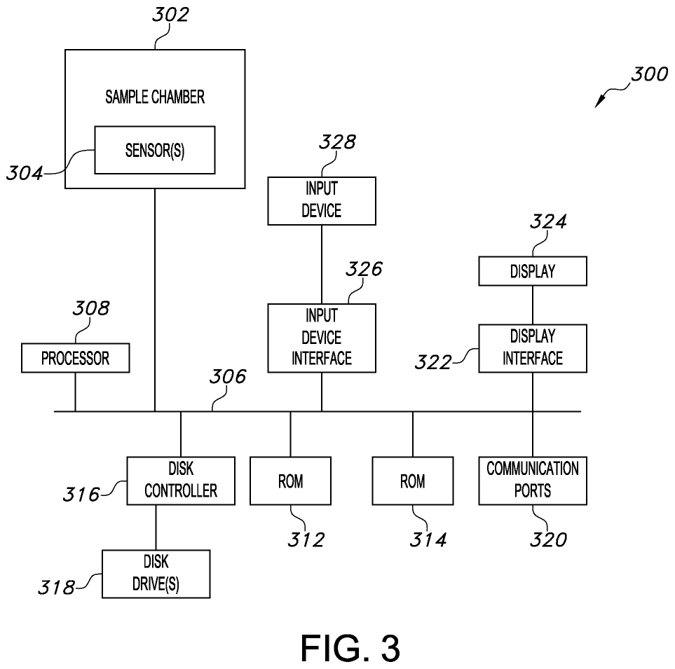

FIG. 3 is a diagram 300 illustrating an architecture of a sample QCM instrument for implementing various aspects described herein. A sampling chamber 302 can include one or more piezoelectric sensors 304 (such as those illustrated in FIG. 1). The medication to be characterized can be flown within the sampling chamber over the piezoelectric sensors 304 such that the resulting resonance changes of a resonating QCM sensor can be detected and electric signals corresponding to such resonance changes (as detected by the instrument) passed to a bus 306. The bus 306 can serve as the information highway interconnecting the other illustrated components of the hardware. A processor 308 (e.g., a CPU, GPU, etc.), can perform calculations and logic operations required to execute a program. A non-transitory processor-readable storage medium, such as read only memory (ROM) 312 and random access memory (RAM) 314, can be in communication with the processing system 308 and can include one or more programming instructions for the operations specified here. Optionally, program instructions can be stored on a non-transitory computer-readable storage medium such as a magnetic disk, optical disk, recordable memory device, flash memory, or other physical storage medium.

In one example, a disk controller 316 can interface with one or more optional disk drives 318 to the system bus 304. These disk drives 318 can be external or internal floppy disk drives such as external or internal CD-ROM, CD-R, CD-RW or DVD, or solid state drives. The system bus 304 can also include at least one communication port 320 to allow for communication with external devices either physically connected to the computing system or available externally through a wired or wireless network. In some cases, the at least one communication port 320 includes or otherwise comprises a network interface.

To provide for interaction with a user, the QCM instrument can include a display device 324 (e.g., LED or LCD monitor, etc.) for displaying information obtained from the bus 304 via a display interface 322 to the user and an input device 328 such as keyboard and/or a pointing device (e.g., a mouse or a trackball) and/or a touchscreen by which the user can provide input to the computer. Other kinds of input devices 328 can be used to provide for interaction with a user as well; for example, feedback provided to the user can be any form of sensory feedback (e.g., visual feedback, auditory feedback by way of a microphone, or tactile feedback); and input from the user can be received in any form, including acoustic, speech, or tactile input. The input device 328 can be coupled to and convey information via the bus 304 by way of an input device interface 326.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

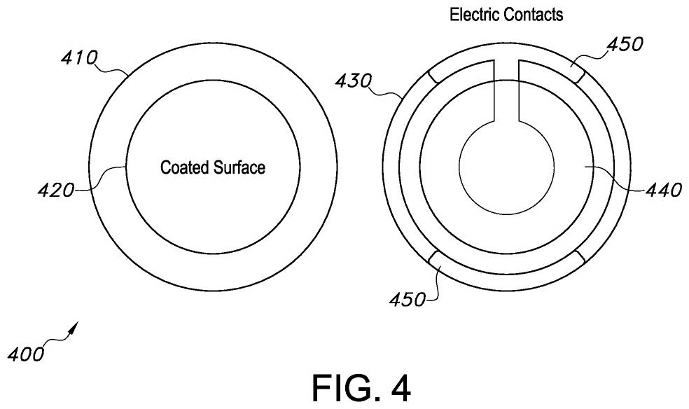

FIG. 4 is a diagram 400 illustrating a top surface 410 of a QCM sensor which may include a coated portion 420 and a back surface 430 of the QCM sensor which may or may not also include a coated portion 440 and, in addition, can include electric contacts 450. The QCM sensor can be an acoustic sensor which can be a resonating piezoelectric A-T cut quartz crystal. As described in further detail below, a surface of the QCM sensor can correspond to or otherwise simulate the surfaces of various receptacles/containers. Further details regarding a QCM sensor as used herein are provided below.

There are a few ways to approach this adsorption problem. Two strategies—changing the structure of protein drugs and changing the concentration of surfactants and other excipients in the drug solution—have both been explored to mitigate therapeutic adsorption and subsequent dosing inaccuracies or loss of protein or protein function. Changing the drug solution environment to account for exclusion effects and other effects and forces a protein has been known to experience is crucial to maintaining protein structure and thus optimizing the desired interactions of the drug with other materials and receptors. For instance, the stabilizing effects of commonly used surfactants polysorbate 20 or 80 (PS20, PS80) are well known. Reduced interfacial affinity of the protein towards either the air-liquid, or liquid-solid interface due to blocking properties of these or other surfactants is the main supposed mechanism of drug adsorption prevention. Surfactants are subject to minimal preferential exclusion effects and have a higher affinity for the interface due to their amphoteric nature and molecular properties.

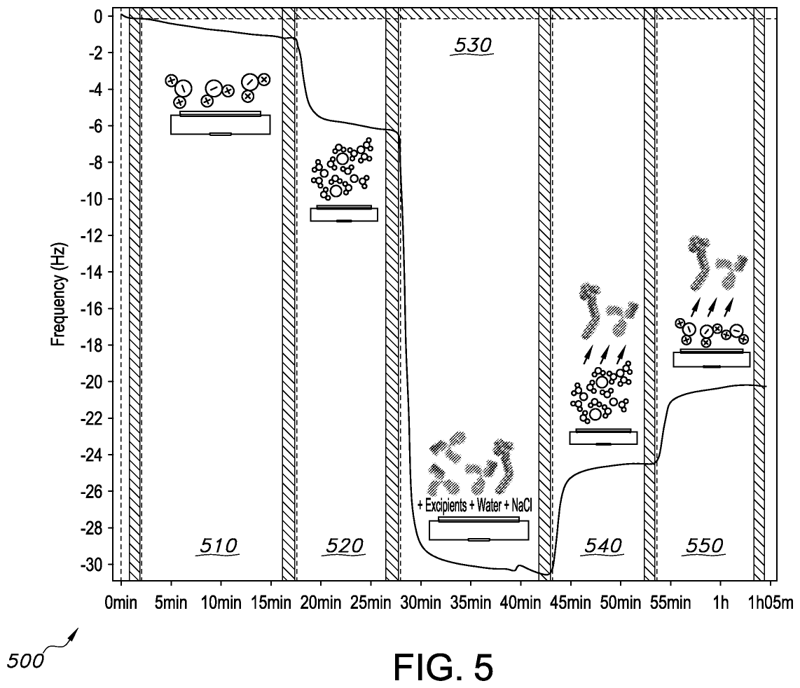

FIG. 5 is a diagram 500 that illustrates an experimental run of a QCM instrument to determine mass adsorbed at a QCM sensor surface which can have a polymeric surface (e.g., a hydrophobic polymer coating). From periods left to right, separated by dashed lines: water baseline period 510, diluent (e.g., 0.9% sodium chloride or normal saline [NS]) baseline to account for affect diluent (e.g., 0.9% sodium chloride or normal saline [NS]) has on resonance 520, sample period in which various solutions mimicking the formulations used in parenteral drug administration were introduced for measurement of adsorption 530, diluent wash off period to determine reversible binding and cleaning of the sensor surface 540, water wash off period to determine reversible binding and cleaning of the sensor surface 550. Sample periods can contain protein with various formulation excipients as a solution either with or without surfactant diluted in diluent or contain no protein with formulation excipients solution but with surfactant all of which simulates and creates conditions for measurement of the therapeutic's interaction with the surface. The frequency measurements can be converted to mass data (e.g., ng/cm2) as described in further detail below. It will be appreciated that while the current subject matter refers to specific diluents such as normal saline (NS), the current subject matter is applicable to a wide variety of diluents. It will also be appreciated that while the current subject matter refers to specific surfactants, proteins, and diluents, such as polysorbate 20, antibodies, or normal saline, the current subject matter is applicable to other surfactants, therapeutics/therapeutic formulations (e.g. other molecular entities being given parenterally, proteins, etc.), diluents, and surface compositions. Assuming the protein or other therapeutic is in an optimized solution for minimizing interfacial stress, aggregation and adsorption still may occur which could affect patient dose and immunogenicity. These concerns manifest especially when protein drug solutions have a large surface area of many polymers in their fluid path to interact with when administered or the dose of the therapeutic is small, or especially both. Studying these interactions is important to patient safety and dose accuracy. Aside from altering the formulation or therapeutic, a few studies have characterized mab interactions and orientations when exposed in solution with and without surfactant to hydrophobic or other surfaces using different techniques including QCM-D.

With the models provided herein, translational surface interaction QCM knowledge is bridged to provide to clinical and formulation development significance. With the current subject matter, qualitative knowledge, and sometimes quantitative knowledge of an investigational Immunoglobulin G (IgG) protein drug's behavior when a sample formulation is tested via QCM-D on different polymer surfaces and when correlated with ECLIA results obtained during the formulation design process to build a model to predict adsorption and loss behavior over a wide range of concentrations. The adsorption estimates over a wide concentration range were experimentally determined for a 100 mL and 250 mL IV bag and IV line and also for different syringes, but other administration setups could be used. It is by measuring the adsorption dynamics using QCM-D that qualitatively and sometimes quantitatively backed decisions on polymers in the supplies used in clinical administration can be made based on adsorption data, and modeling of adsorbed mass loss can be done to predict behavior for a specific drug and its formulations or dilutions containing a wide range of protein drug concentrations.

The advances provided herein were experimentally validated.

General Formulation Materials. General formulation materials were used including glacial acetic acid (99%), ethylenediaminetetraacetic acid (EDTA), sodium acetate trihydrate, sucrose, surfactant, methionine, and sodium chloride. In addition, the investigational IgG proteins used in both purified, preformulated bulk form as well as fully formulated form were obtained.

QCM Materials. Polypropylene and polyvinyl chloride sensors, as well as an associated automated QCM instrument were utilized. Pipettors and balances as well as falcon tubes for solutions were obtained. Deionized, filtered water was used for all solution preparation. Cleaning liquids for the sensors and the instrument were 100% ethanol, 2% sodium dodecyl sulfate (SDS), deionized and filtered water, and Deconex 11.

Experiment Materials. Materials that were characterized including a polyvinyl chloride (PVC), polyethylene (PE), or polypropylene (PP) IV administration set, a polyvinylidene fluoride (PVDF) or polyethersulfone (PES) inline filter, a PE, PP, or PVC IV bag, and a PP or polycarbonate (PC) syringe. In addition, a polyethylene terephthalate glycol (PETG) bottle was used to collect infusate, and sample solution falcon tubes were also used. Other equipment included a plate reader, a plate shaker, a plate washer, pipettors, sterile liquid vials, and pipette tips. ECLIA assay buffer and other solutions prepared the day of mock infusion sampling. Experimental solutions and materials prepared in-house included 10% saline and assay buffer, standard analogous antibody for comparison to samples, the high, medium, and low-quality control investigational IgG protein, wash buffer, biotinylated specific antibody receptor ligand, and assay buffer. Further, a ruthenium-R10 reagent was used in assays in addition to cell culture grade water, read buffer, and streptavidin-coated gold plates. When ECLIA was not used for protein dose quantification, Protein A HPLC immunodetection columns were used for quantification of amount of protein in solution when dosed.

The experiments detailed below were informed using quartz sensors coated with various surface compositions such as PVC, PE, PES, PVDF, PC, or PP. These sensors can be pre and post-run cleaned, for example, by way of a 30 min soak in 1% Deconex 11 Solution, a minimum 2 hr soak in DI water (usually overnight), followed by a rinse with DI water and 99% ethanol three times and then blown dry by medical grade nitrogen gas. The sensors were then inserted into a QCM unit as was sample solution, diluent (e.g., NS), and water. Runs were configured and data and procedures were collected. Experimental data was then transformed from frequency to mass data using, for example, the above equations. Measurement of frequency and dissipation occurred as follows generally for all runs during each step (and subsequently defined period) with all flow rates for every liquid set at 10 μl/min (also illustrated in FIG. 5):
Period 1 (510)—Establishment of baseline in water (priming sequence˜5 minutes+10 minutes).
Period 2 (520)—Establishment of baseline in normal saline (15 min).
Period 3 (530)—Sample solution added and run over sensor (10 min).
Period 4 (540)—Washing with dilluent (10 min).
Period 5 (550)—Washing/cleaning of system with water (10 min+probe and sample port cleaning sequence).

Steps can be followed to clean the sensor and QCM instrument post-run as per manufacturer procedures (period 6, not shown in figure).

In an example involving protein 1 (referenced below), the sample solutions in period 3 are one of several possibilities (both listed or not listed herein) in any one run: fully formulated investigational drug product (IP) diluted in a diluent (e.g., NS, etc.) with a surfactant (e.g., PS20, etc.) and all other excipients and protein drug, fully formulated IP diluted in a diluent without PS20 but with all other excipients and protein drug, or fully formulated IP diluted in diluent with PS and all other excipients but no protein drug. Each sample run experiment sequence can be performed multiple times for each 6-step run sequence, and the average mass of all runs at a given condition (determined using equation 2) can, as an example, be used as the mass for estimation in the variables in equations 3 and 4. The sample solutions, if they contained protein (e.g., protein 1) in the corresponding runs that did, were in one example, dilutions of a stock IP solution to concentrations of 0.1 mg/mL, 0.01 mg/mL, 0.001 mg/mL, and 0.0001 mg/mL. It will be appreciated that other concentrations or solutions can be utilized according to the IP presentation in the clinic, and in other data presented, differed. This example serial dilution was at four levels often seen in clinic of the formulation diluted with diluent, and the corresponding dilutions occurred with the solutions containing only formulation excipients with PS20 but no protein to the corresponding concentrations to the four listed above. With these experiments, normal saline, along with the above formulation solutions, were made the day of corresponding experimental QCM runs using deionized water. USP <797> aseptic technique was used when preparing solutions to mimic a hospital preparation environment.

In one experimental run, over 60 sensorgrams (i.e., the outputs of the QCM-D instruments) were analyzed and transformed from frequency to mass adsorption data in the aforementioned example. Here the mass adsorbed during the sample period was of primary interest, and the mass during this period was measured by subtracting the average mass recorded and calculated during the diluent period where an ionic liquid had effect on resonance (period 2 above/520 in FIG. 5) from the average mass shift recorded and calculated during the sample period (period 3 above/530 in FIG. 5). Mass was determined in this manner for all three below variables in equations 3 and 4 (above) for each run and then the masses were averaged together for each variable. At each concentration and for each material, for this example experimental run, an average adsorbed mass in the three separate conditions with the three above defined solutions over all available runs during the sample period was determined (period 3 above/530 in FIG. 5). These adsorbed masses were then compared against each other within the same and between different materials at the same and different concentrations as per equations 3 and 4.

In one experimental example, the different conditions and adsorbed masses were used to make an estimate of both mass composition at the adsorbed surface of protein in ng/cm2 (equation 3 above) and mass composition at the adsorbed surface of surfactant in ng/cm2 (equation 4 above) when protein and polysorbate were both exposed simultaneously to the hydrophobic polymer surfaces. In both equations, x is the measured adsorbed mass in ng/cm2 when fully formulated IP diluted in NS without surfactant but with all other excipients and protein drug is sampled via QCM, y is the measured adsorbed mass in ng/cm2 when fully formulated IP diluted in NS with surfactant and all other excipients but no protein drug is sampled via QCM, and z is the measured adsorbed mass in ng/cm2 when fully formulated investigational drug product (IP) diluted in diluent (e.g., NS, etc.) with surfactant (e.g., PS20, etc.) and all other excipients and protein drug is sampled via QCM.

The masses in equation 3 were taken from the average measurement of the Sauerbrey-transformed frequency shift during each sample period. The masses estimated using equation 3 were then correlated with solution protein concentration and the amounts lost at the same concentrations of drug product in NS IV bags from the ECLIA-assayed infusion experiments and a natural log-linear function model was developed to predict loss of drug results at a wider range of concentrations for administration materials. The estimates over a wide concentration range were determined for a 100 mL and 250 mL IV bag and PVC IV line. The model predicted loss based on a sample of actual bag volume fills as the bag volume can vary by a set number of mL around the nominal amount specified on the bag. Also, a natural log-linear model was developed to relate concentration to adsorbed amount. In a small number of experiments, the QCM-measured adsorbed mass (which is not a true mass, but rather the liquid effects of the solution) in ng/cm2 when fully formulated IP diluted in NS without PS20 or protein drug but with all other excipients was compared to NS period 2 (operation 520 in FIG. 5) as described above in order to verify the mass adsorbed at the sensor surface was in fact composed of almost entirely PS20 or protein when adsorption was observed when experiments were conducted.

To further validate the advances herein, infusion experiments were conducted in 100- and 250-mL NS IV bags with attached administration sets. The full IP formulation containing drug and PS20 as well as the other included excipients were diluted by admixture into the bag it was to be tested in in an ISO Class 5 vertical laminar flow hood using USP <797> aseptic techniques for sterile drug preparation. The bags were left at ambient room temperature and light for a 24-hour period, then infused into PETG bottles and samples were drawn up and diluted 1:10 in ECLIA assay buffer.

ECLIA assay, samples, and wash buffers were prepared the day of the experiment. The ECLIA active protein content method was a sandwich immunoassay based on capture by the receptor ligand and detection with a generic antibody detection reagent utilizing electrochemiluminescence. A streptavidin coated plate was loaded with receptor containing modified biotin, then standard curve calibrators for a 10-point standard curve were added and the points established, quality controls were run for concentration comparison, then diluted samples were added. After incubation, the assay plate was washed, and the fluorophore-labeled detection reagent was added to the assay plate. Following incubation, the assay plate was washed and then read on a plate reader after addition of read buffer. The active concentration of the quality controls and samples is then determined by interpolation from the standard curve. Duplicate samples were run and allowance of ±20% variation was standard for the developed method for each and between each sample. Data was then analyzed for variance and internal standardized acceptance criteria.

With one set of experiments, the results for percent recovery as measured were then compared with the original solutions' concentrations. Unacceptable results via ECLIA were defined as ≥30% of dose lost difference from admixture of nominal concentration and the infusate collected in the PETG bottle. The NS bags used for IP preparation were weighed before and after admixture as well as post-infusion. This allowed controlling for the specific fill volumes of each individual IV bag used and the exact concentration of IP preparation this corresponds to, which were very close to the nominal concentration levels being tested between 0.1-0.0001 mg/mL. The same size IV bags of the two sizes tested and the same type of IV line used in experiments were deconstructed and measured for internal fluid path surface area. Information for surface areas were then verified with manufacturers. The results of the percent recovery studies were compared with QCM results.

### Experimental Results

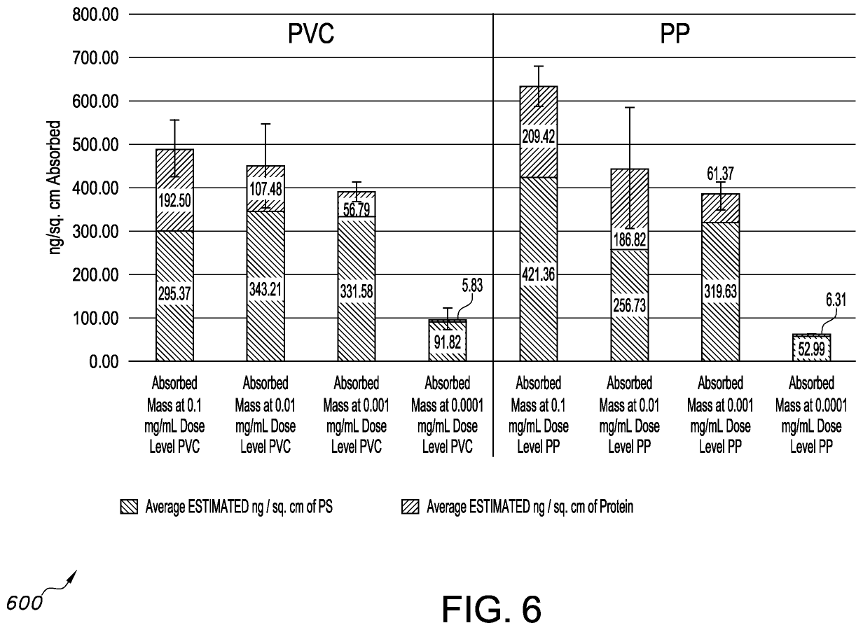

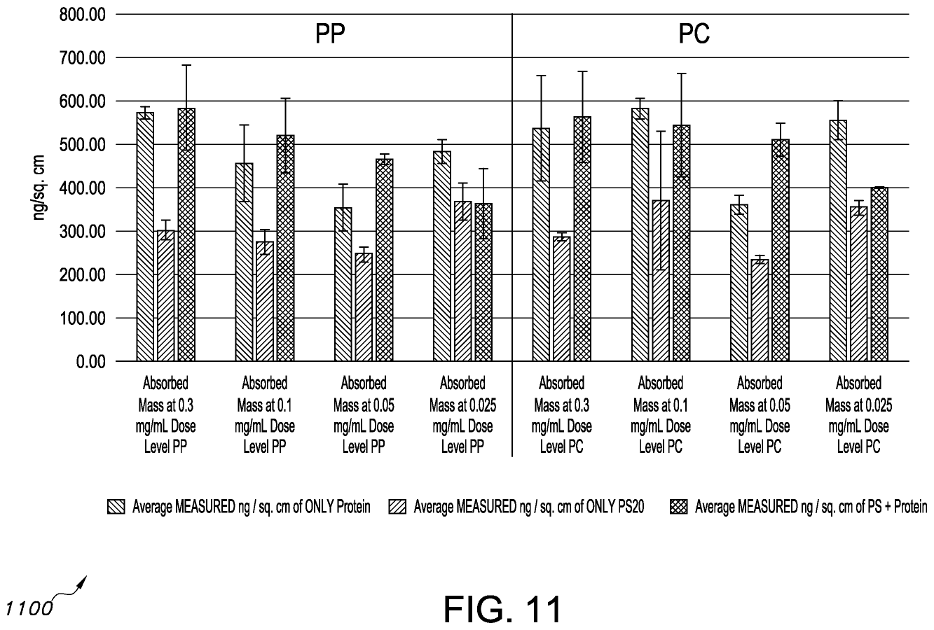

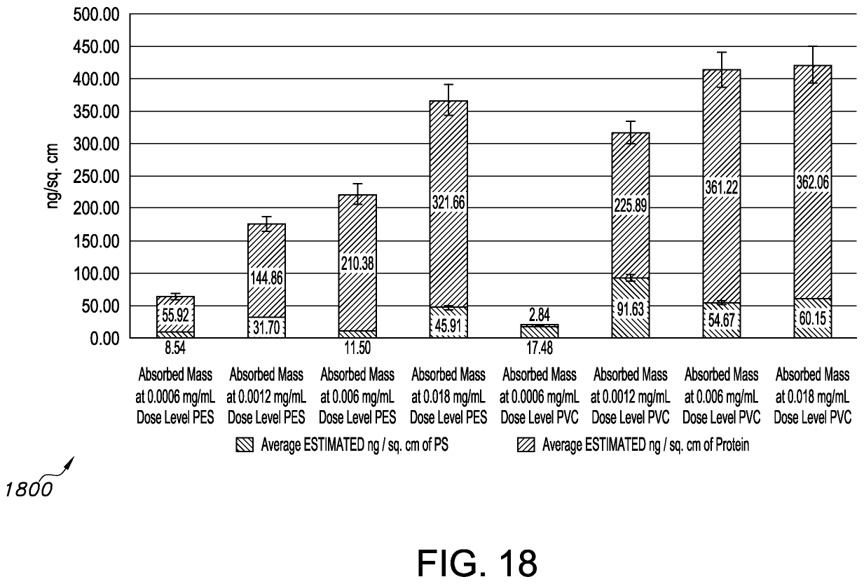

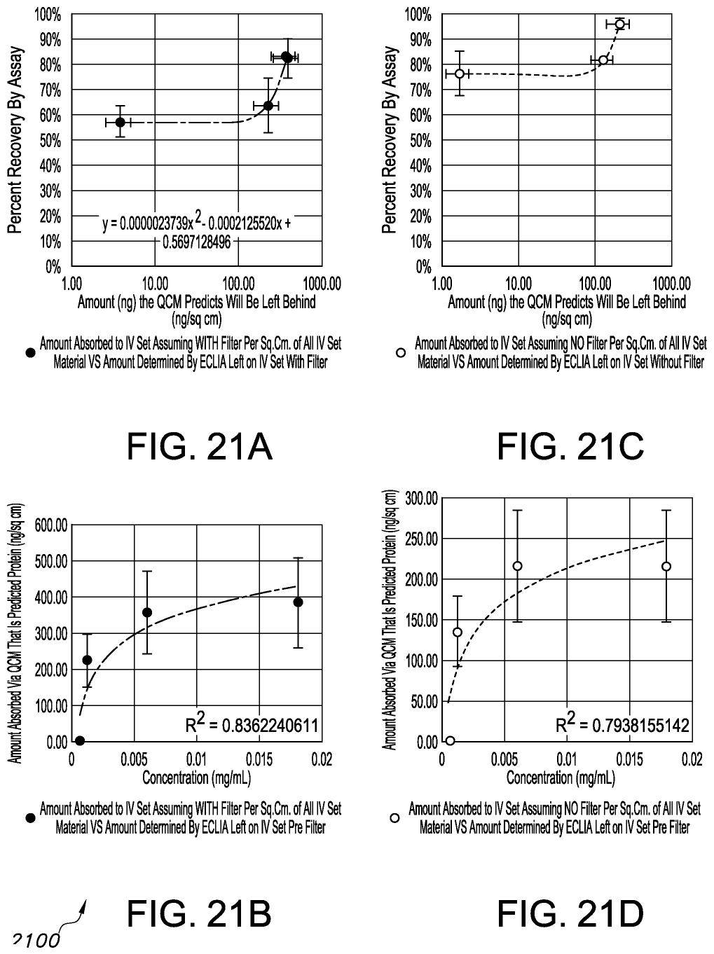

The results of the experiments for a first protein (referred to herein as protein 1) are summarized and illustrated in FIGS. 6 to 10. The results of the experiments for a second protein (referred to herein as protein 2) are summarized and illustrated in FIGS. 11-17. The results of the experiments for a third protein (referred to herein as protein 3) are summarized and illustrated in FIGS. 18-20. The results of experiments for a fourth protein (referred to herein as protein 4) are summarized and illustrated in FIGS. 21A-D, 22-24, 25A-D and 26.

Protein 1 Experiments. On average, the percent of the total mass that is estimated to be protein (i.e., protein 1) adsorbed at all concentrations when the adlayer and sample period solution was made up of both surfactant and investigational IgG protein exposed to the surface simultaneously was 25.54% [95% CI±14.6%] of the mass for one of the polymers and 23.10% [95% CI±11.8%] of the mass for one of the polymers. Similar adsorption patterns between the polymers were seen at all masses in all conditions. Slightly more protein (i.e., protein 1) was estimated to be adsorbed at all concentrations for PP but not to an appreciably large amount. A large drop off in masses adsorbed when PS20 and protein was exposed to the hydrophobic surfaces simultaneously was observed between the 0.001 mg/mL and 0.0001 mg/mL sample IP concentrations.

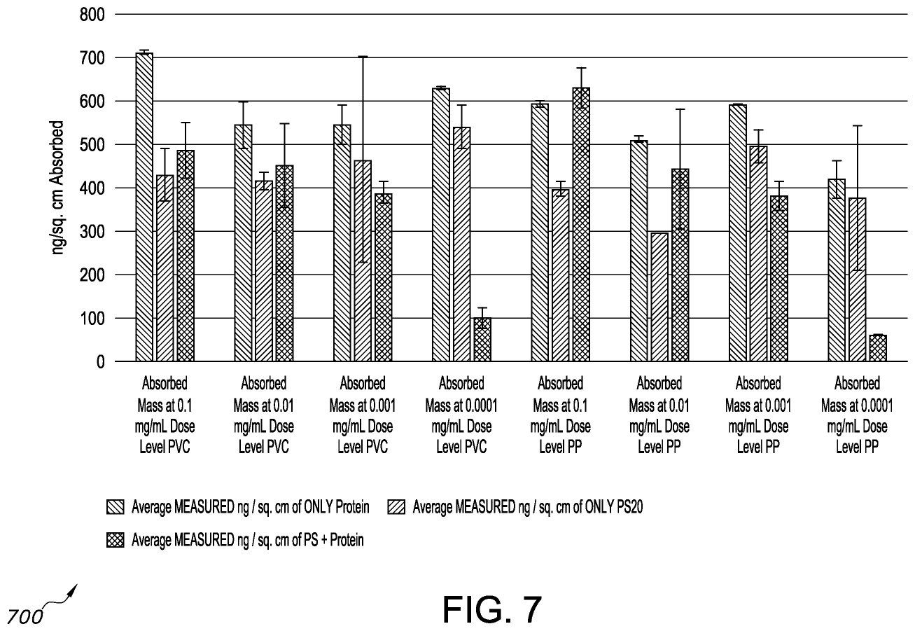

FIG. 6 is a diagram 600 that illustrates estimates of mass contributions of surfactant and protein to layer at polymeric sensor surfaces at different concentrations for protein 1. Each set of four bars from left to right was the total mass adsorbed at 0.1 mg/mL (10 mg dose), 0.01 mg/mL (1 mg dose), 0.001 mg/mL (0.1 mg dose), and 0.0001 mg/mL (0.01 mg dose) on either PVC (right four bars) or PP (left four bars) split out into the estimated mass contributions by color. Each dose calculated assuming drug was admixed to the four concentrations tested using a 100 mL NS IV bag for dilution and subsequent infusion. Error bars are included for the total mass adsorbed when both surfactant and investigational IgG protein were exposed to the surface during the sample period. All run average masses for protein with formulation excipients solution either with or without surfactant diluted in NS or formulation excipients solution without protein but with surfactant can be seen in diagram 700 of FIG. 7.

FIG. 7 is a diagram 700 illustrating measurements of adsorbed masses of only investigational IgG protein, only surfactant, and investigational IgG protein+surfactant in formulated solution diluted in NS. The average adsorbed amounts in each condition for the experiments took into account the NS effect and period by subtracting it from the sample period mass which is shown here. These measured average amounts were used in equations 3 and 4 to create estimates of how much each substance contributed to the mixed adsorbed layer when the solution with both surfactant and investigational IgG protein in the formulated solution diluted in NS were exposed to the hydrophobic polymers. The left four bar groupings relate to one of the polymers the right four are for other polymers.

The individual masses adsorbed to each surface are shown in FIG. 7. These masses were used as shown in equations 3 and 4 to estimate mass fractions in surfactant and investigational IgG protein in the same solution. In every case, the average mass recorded of adsorbed protein in solution diluted in NS without surfactant was very close to the average mass recorded when surfactant in the formulation solution diluted in NS was sampled. The average mass recorded when both surfactant and investigational IgG protein were in the formulation solution diluted in NS was always close to the average mass recorded when just protein in the formulation solution diluted in NS was sampled for protein 1. When surfactant and investigational IgG protein were sampled in solutions, the average mass was dependent on the concentration of the investigational IgG protein component of the solution, and while the 0.1 mg/mL and 0.0001 mg/mL solutions had a large difference in measured average adsorbed mass, the 0.01 mg/mL and 0.001 mg/mL measured average adsorbed masses were more similar. Another important result was that the behavior of all masses adsorbed at each corresponding concentration between materials was the same when comparing which masses were greater or lesser than the other masses adsorbed (i.e. if the surfactant only mass was lesser than the surfactant and investigational IgG protein mass but also lesser than the investigational IgG protein only mass at 0.1 mg/mL one of the polymers, the same exact pattern was observed at the other polymer 0.1 mg/mL level, and the same goes for most other concentration patterns like this). This creates concentration and material mass signatures.

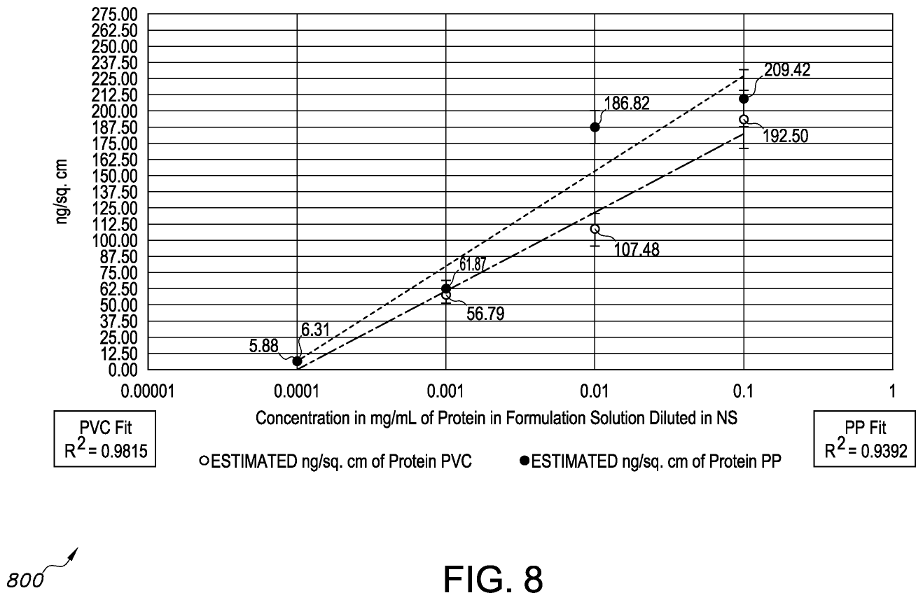

FIG. 8 is a diagram 800 that illustrates concentration of protein in solution vs. estimates of mass contributions of investigational IgG protein to adsorbed layer at one of the polymers and other polymer sensor surface. As is illustrated, it was found that there is a positive concentration relationship of investigational IgG protein in solution and adsorbed investigational IgG protein. A natural log-fitted function was plotted as a line of best fit for both materials. Points were labeled with the estimated adsorbed amounts. The error bars were constructed based on 95% CI for fraction of protein adsorbed when surfactant and investigational IgG protein was exposed to the hydrophobic surfaces simultaneously.

The amount of estimated adsorbed investigational IgG protein was observed to be dependent on the concentration of investigational IgG protein in the sample solution, and this can be seen in FIG. 8. A natural log-linear fit yielded a coefficient of determination greater than 0.9 for both polymers, indicating the concentration of investigational IgG protein in solution (and by extrapolation the surfactant as well) explains the variation in adsorbed amounts. Again, the one of the polymers' adsorbed amounts were observed to be estimated at a slightly higher value in FIG. 8, however not to a large amount. It was found that extrapolation further into lower and lower concentrations does yield a best fit function estimate between the two fits at some low concentration that is the same estimated adsorbed amounts of investigational IgG protein, as is seen as well in the lowest concentration level adsorbed mass estimates being within one nanogram of each other.

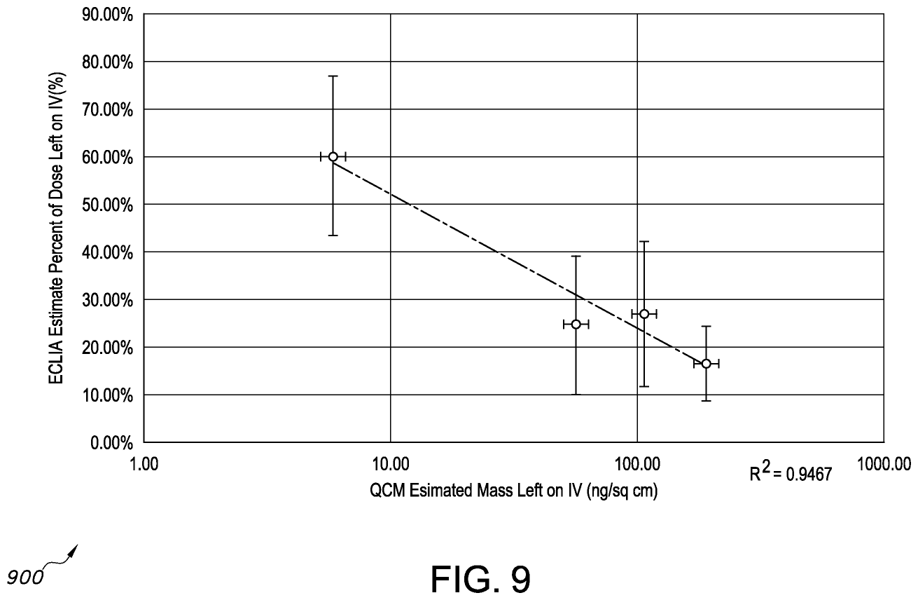

FIG. 9 is a diagram 900 illustrating ECLIA-measured percent of dose lost on an IV Set vs. QCM estimated mass left on the IV Set. As is illustrated, there was a negative relationship between amount of investigational IgG protein adsorbed per square centimeter via QCM experiments and percent of dose lost on IV set measured directly by ECLIA. A natural log-linear function was the line of best fit. These results indicated that the larger the dose, the less fraction of it is lost on the IV set, highlighting the need for low-dose knowledge of adsorbed amounts. High variability in the ECLIA method existed due to the assay not being completely optimized.

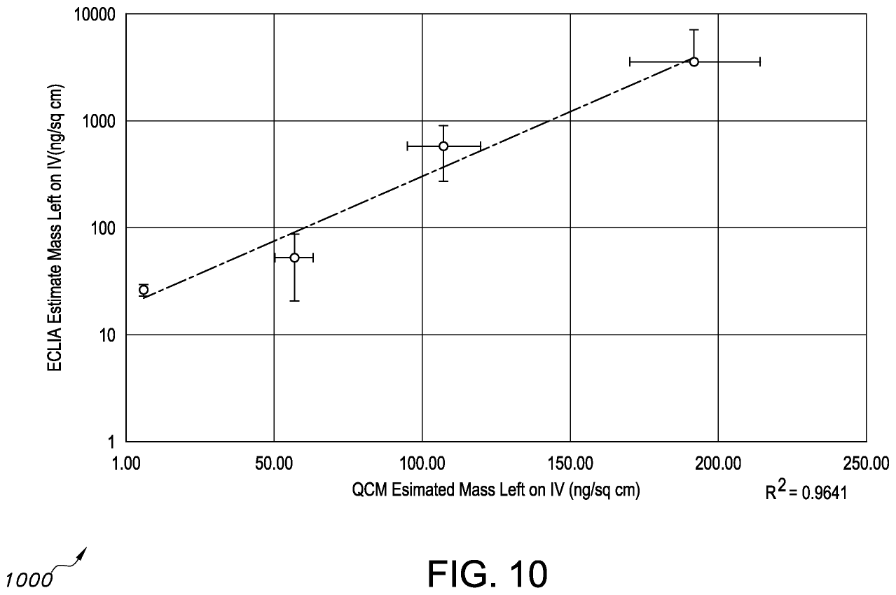

The estimated amounts adsorbed as they relate to ECLIA-assayed infusion study results are illustrated in FIGS. 9 and 10. The negative relationship in FIG. 9 between estimated mass of protein left on the IV set via QCM and ECLIA-estimated percent of dose left on IV set shows the result of the hypothesis of the effect dose size has when the formulated therapeutic solutions diluted in NS at different concentrations were all exposed to the same environment and, by extension, square centimeters of fluid path in the IV line. The higher the dose, the less the tiny fraction of drug estimated to be left on the IV set changed the overall dose by an appreciable and therapeutically relevant percentage. The higher dose concentration solutions sampled also had, when compared to the lower dose level concentration solutions sampled, one or two orders of magnitude higher concentrations of surfactant and investigational IgG protein in solution.

FIG. 10 is a diagram 1000 that illustrates ECLIA estimated mass left on a polymeric IV set vs QCM estimated mass left on the polymeric IV set. As is illustrated, there is a positive relationship between the estimated amount lost on the IV set and the ECLIA estimated amount lost on IV set is shown here. A natural log linear-fitted function was plotted as a line of best fit. The ECLIA estimate was based on percent recovery results from studies as assayed by ECLIA active protein content methods. Percent lost was calculated by comparing the concentration submitted for ECLIA testing, and the ECLIA assay result, then that percentage was used to estimate how many nanograms of investigational IgG protein were left on the IV set. The negative Y-axis error bar for the rightmost point is not shown because it is below 0. High variability in the ECLIA method exists due to the assay not being completely optimized.

FIG. 9 is analogous to FIG. 10, as the percentages, the exact volumes of the NS IV bags, and the doses at the corresponding sample solution concentrations from FIG. 9 were used in FIG. 10. The percentages and infusion volumes and conditions were used to estimate the nanograms per centimeter of fluid path that would have to had been lost to the IV set in the ECLIA-assayed infusion experiments during infusion. This yielded the positive correlation between the QCM estimated adsorbed amounts of protein drug and the ECLIA assayed infusion masses drawn from the percent of dose lost which corresponds logically with FIG. 9. Both sets of data taken together, the larger the dose the less percent of dose lost to adsorption on the IV set, and the QCM estimates of this adsorption correlate well to infusion performance as measured by ECLIA. These two sets of data correlated with QCM estimate data yield relationships with lines of best fit with high coefficients of determination.

It is noted that these results were based on calculations and estimates that use real world experimental data that is very basically processed. The ECLIA results measuring infusion performance did not correspond 1:1 to the adsorbed estimate masses found in QCM at the corresponding concentrations. This then led to correlating the data, and the relationships found however were very strong correlations between results of both experiments. This data primarily considered solely adsorption behavior of the investigational IgG protein and surfactant only using simple equations using a large amount of QCM experimental data.

A final small amount of experiments were performed to verify whether the other excipients in the formulated solution diluted in NS were appreciably different than the normal saline solution. During the runs, the effect on frequency and by relation mass data was tested by running as a sample solution the formulation minus surfactant or investigational IgG protein, diluted to the same concentration as the corresponding surfactant and drug-containing solutions. The mass shift transformed from frequency the NS only periods produced on average over all NS periods during and right before the sample period containing investigational IgG protein or surfactant or both was 63.59 ng/cm2 [95% CI±5.69 ng/cm2] versus the formulated solution minus the investigational IgG protein and surfactant in NS at 82.25 ng/cm2 [95% CI±8.03 ng/cm2]. There is only at most 32.37 ng/cm2 separating these estimates when it comes to the confidence intervals, which is a negligibly small amount of mass, and by relation frequency shift.

FIG. 11 is a diagram 1100 that illustrates a different experiment in connection with measurements of adsorbed masses of only protein, only surfactant, and protein+surfactant in a formulated solution diluted in diluent. The diagram 1100 illustrates average adsorbed amounts in each condition for the experiments considering the diluent by subtracting it from the sample period mass. These measured average amounts were used in equations to create estimates of how much each substance contributed to the mixed adsorbed layer when the solution with both surfactant (e.g., PS20, etc.) and protein in the formulated solution diluted in diluent (e.g., NS, etc.) were exposed to the hydrophobic polymers. The left four bar groupings were for PP, the right four were for PC.

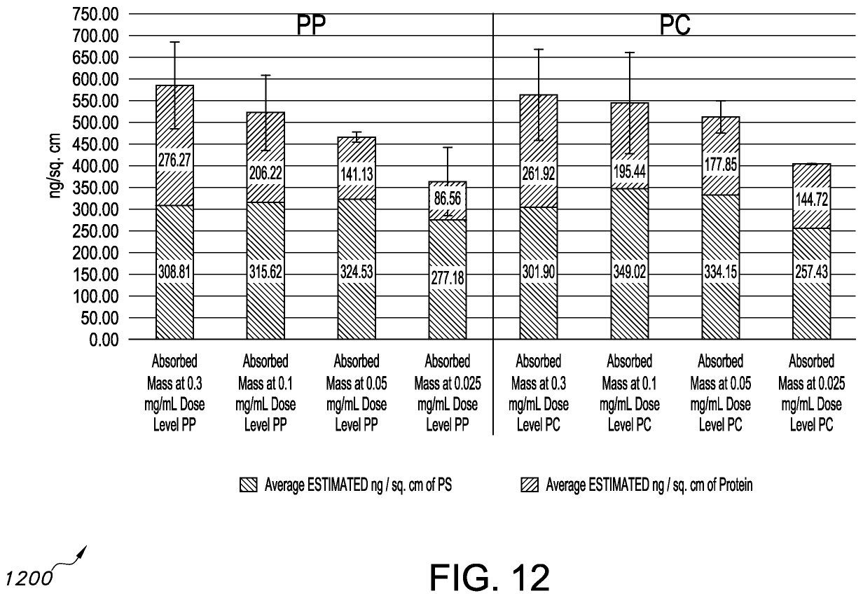

Protein 2 Experiments. FIG. 12 is a diagram 1200 that illustrates estimates of mass contributions of surface and the protein 2 to layer at PP and PC sensor surfaces at different concentrations. With this diagram 1200, each set of four bars from left to right was the total mass adsorbed at 0.3 mg/mL, 0.1 mg/mL, 0.05 mg/mL, and 0.025 mg/mL on either PP (right four bars) or PC (left four bars) split out into the estimated mass contributions. Each dose was calculated assuming the drug is admixed to the four concentrations tested using diluent and syringes for dilution and subsequent administration. FIG. 12 includes error bars for the total mass adsorbed when both surfactant and protein are exposed to the surface during the sample period. The diagram in particular illustrates run average masses for protein with formulation excipients solution either with or without surfactant diluted in a diluent or formulation excipients solution without protein but with surfactant.

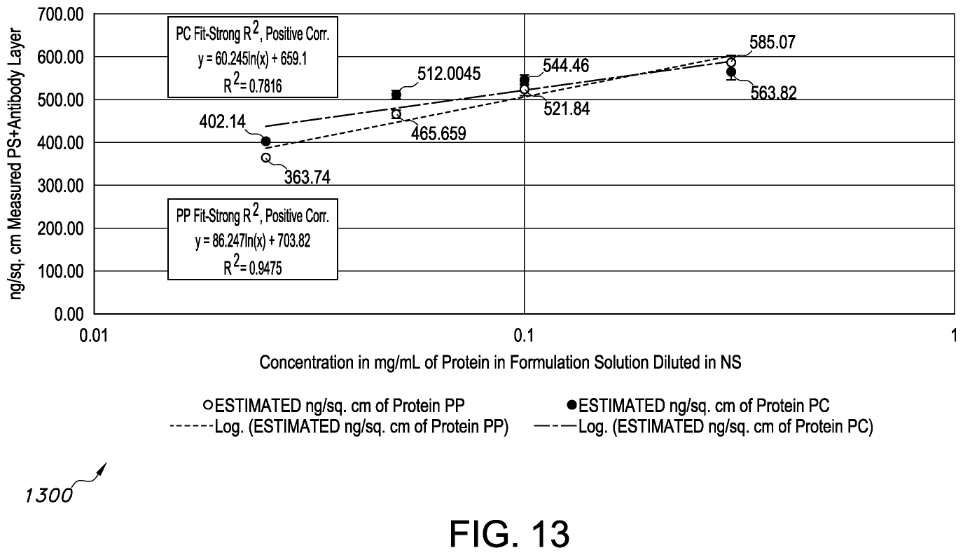

FIG. 13 is a diagram 1300 that illustrates measurements of mass contribution of protein 2 and surfactant in full formulation in diluent at PP and PC sensor surfaces at different concentrations. As this diagram 1300 illustrates, the more protein and surfactant in the same solution, there will be more measurable surfactant and protein adsorption via QCM. This diagram 1300 also illustrates that one can assume that some amount of protein and surfactant adsorb to the polymer surface which can be due, in part, to varying protein solution concentration. As a result, it can be assumed that the mass of both protein and surfactant in what is adsorbed goes down and varies very closely with protein concentration.

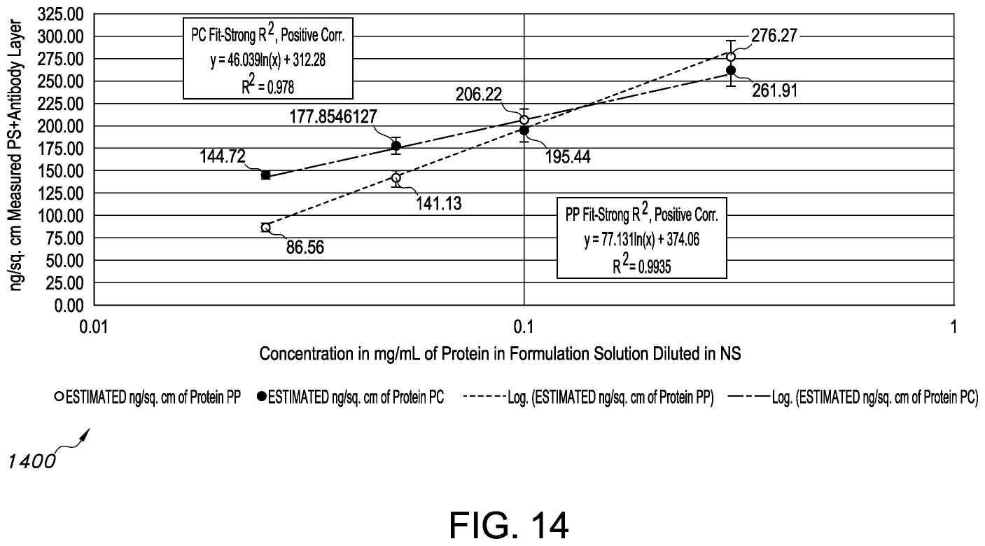

FIG. 14 is a diagram 1400 that illustrates concentration of protein in solution versus estimates of mass contributions of protein to adsorbed layer at PP and PVS sensor surfaces. This diagram 1400, in particular, shows that the more protein and surfactant in the same solution, there will be more estimated protein adsorption via QCM. FIG. 14 also shows a positive concentration relationship of protein drug in solution and adsorbed protein drug. Here, a natural log-fitted function was plotted as a line of best fit for both materials. Points were labeled with the estimated adsorbed amounts. Error bars were constructed based on 95% CI for fraction of protein adsorbed when surfactant (e.g., PS20, etc.) and protein was exposed to the hydrophobic surfaces simultaneously.

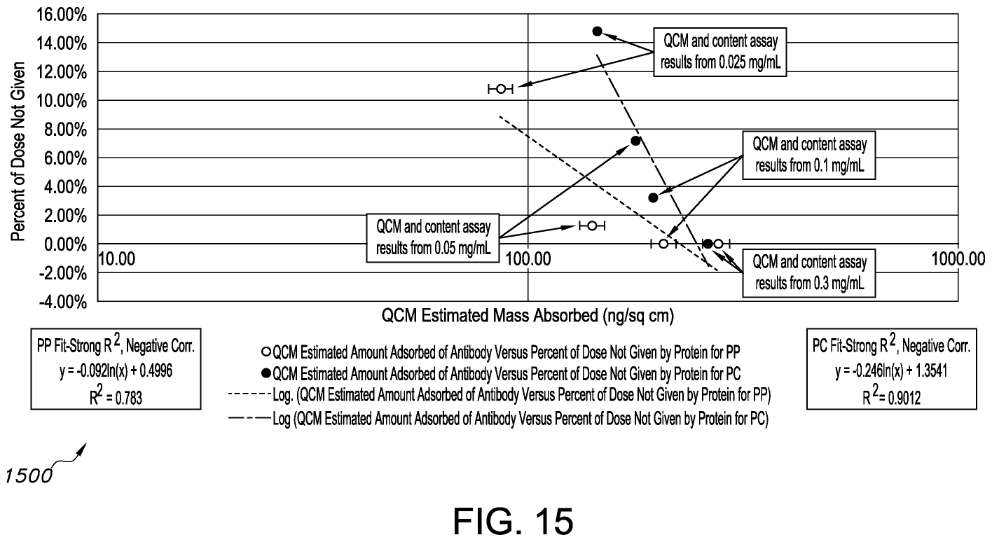

FIG. 15 is a diagram 1500 that illustrates the QCM estimated amount adsorbed of an antibody versus percent of dose not given by content assay for PP and PC sensor surfaces. Such an arrangement may seem counterintuitive, however, even though amount left on the polymer is low and the percent of dose left behind is high, this is because the dose is low, thus a higher percent of dose is left on the polymer. Stated differently, as the dose increases, a less and less substantial portion of the whole dose is left behind, and even though the amount adsorbed is going up, it cannot keep up and be the same portion of the whole dose it was when dose was low. A strong association means that if the QCM results are known, then the dose results are also able to be estimated well using the function that relates the two.

In FIG. 15, there is illustrated a negative relationship between amount of protein adsorbed per square centimeter via QCM experiments and percent of dose lost on a medication container (e.g., IV set) measured directly by content assay. A natural log-linear function was used as the line of best fit. These results indicated that the larger the dose, the less fraction of it is lost on the medication container (i.e., syringe), highlighting the need for low-dose knowledge of adsorbed amounts.

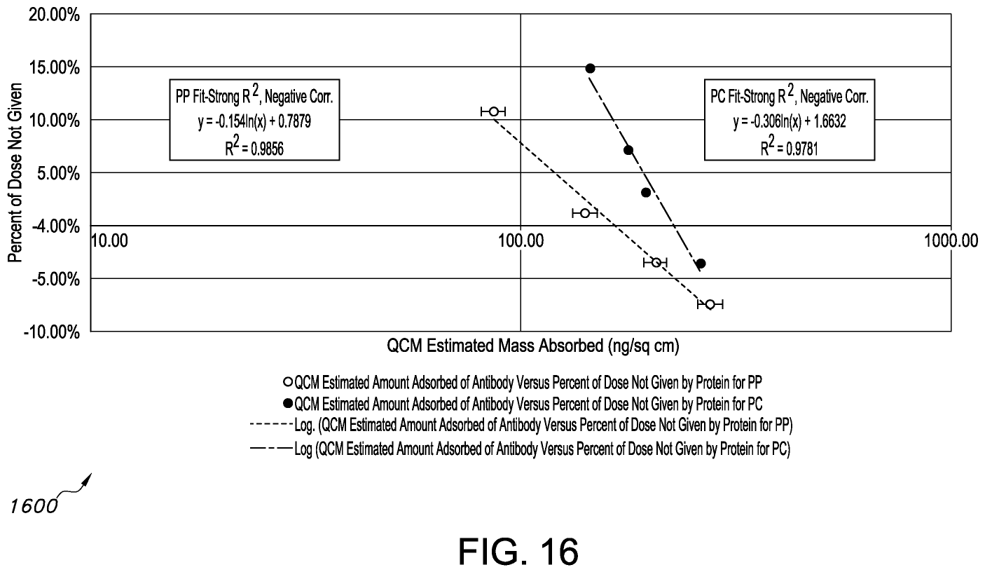

FIG. 16 is diagram 1600 illustrating the QCM estimated amount adsorbed of protein 2 versus percent of dose not given by content assay for PP and PC. These results assumed a 2 mL dose was given for each concentration in a 3 mL syringe drawn to the 2 mL mark with a measured liquid contact surface area of 20.745 square centimeters in the syringe.

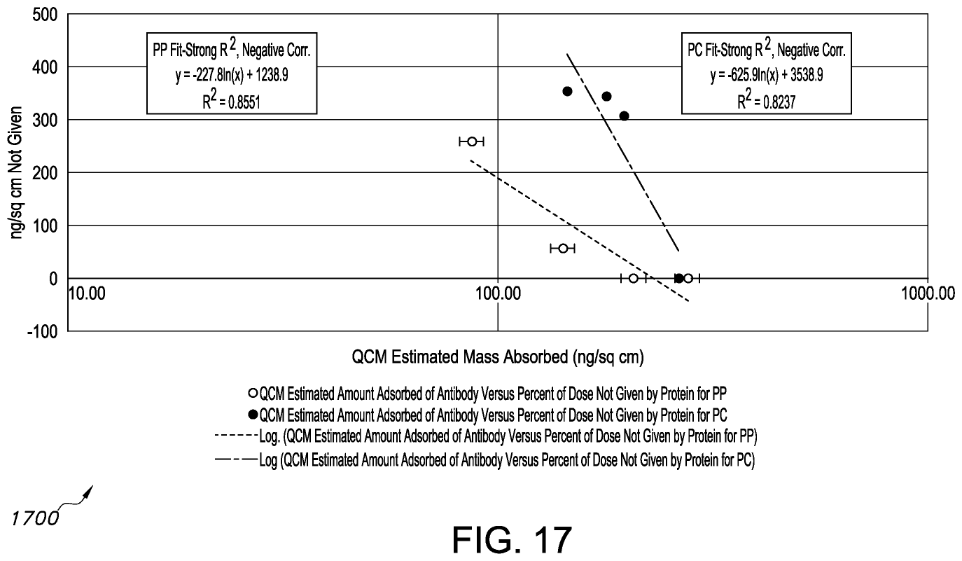

FIG. 17 is a diagram 1700 illustrating the QCM estimated amount adsorbed of protein 2 per area versus mass of dose per area not given by content assay for PP and PC. These results also assumed that a 2 mL dose was given for each concentration in a 3 mL syringe drawn to the 2 mL mark with a measured liquid contact surface area of 20.745 square centimeters in the syringe.

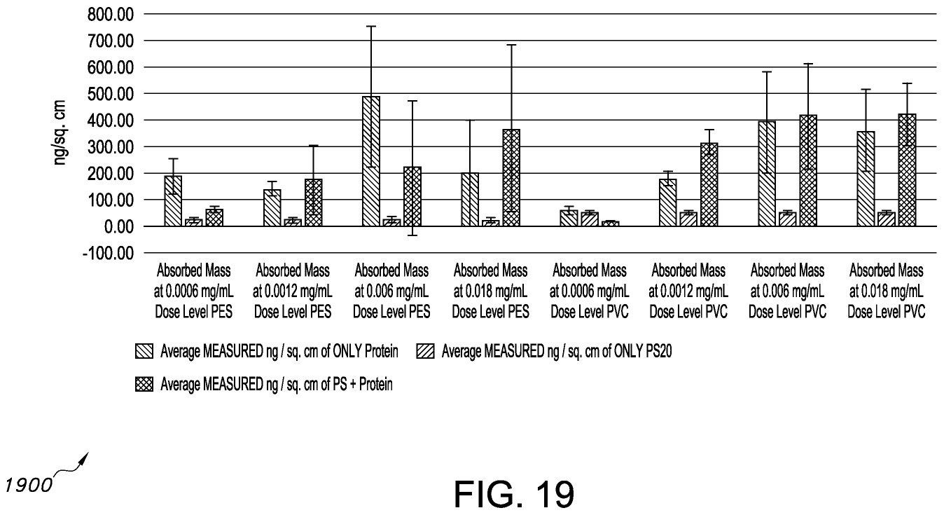

Protein 3 Experiments. FIGS. 18 and 19 are diagrams 1800, 1900 illustrating estimates of mass contributions of PS20 and protein to an adlayer at PVC and PES sensor surface at different concentrations and three-condition average adsorbed masses. FIG. 18 illustrates different adsorbed amounts of protein and PS20 when a PS20-poor solution was flown over a sensor surface of either PES (right four bars) or PVC (left four bars). Unlike when more PS20 is present in solution, there is substantially more protein mass contribution at the adsorbed layer, which is a worst-case scenario for adsorption and aggregation. Adsorbed protein fractions varied with concentration while smaller variation less than 100 ng/sq cm was seen in PS20 fractions. These estimates were constructed using equations 3 and 4 and the adsorbed masses in the PS20 only, PS and protein, and protein only conditions shown in FIG. 19. Error bars were the 95% confidence intervals around the adsorbed mass average in FIG. 19 and the percent of each component's mass average in FIG. 18. The runs with protein had wider intervals due to an increased variance seen between runs with this DP.

Average adsorbed amounts were measured for all three conditions and ECLIA infusion experiments were successful in measuring percent recovery (seen in FIGS. 21A-D) for all conditions of fully formulated drug product. The results of the adsorption experiments are shown in FIGS. 18 and 19. On average, the layer at the surface over both polymers was made up of adsorbed protein contributing 64.44% [95% CI±7.5%] of the mass for PVC and 87.78% [95% CI±6.1%] for PES, and PS20 which was 35.56% [95% CI±33.7%] for PVC and 12.22% [95% CI±5.2%] for PES. Amounts and protein and polysorbate measurements as seen in FIGS. 18 and 19 mostly varied with concentration, while PS20 only runs were all at the same concentration and all similar mass with the average mass contribution being 40.20 ng/sq cm [95% CI±19.77 ng/sq cm]. There were no significant differences between adsorbed masses when comparing materials via a two-sample t-test.

FIGS. 18 and 19 provide some useful comparisons, and generally the amount of protein adsorbed increased with concentration and was substantially lower at the lowest concentration when compared to the other concentrations for both materials. The amount of protein alone adsorbed in protein only runs, as well as the mass fraction of protein in the protein and PS20 runs was always greater than the PS20 masses either alone or the PS20 fraction of mass in the adlayer. The adsorbed mass when protein and PS20 were exposed at the same time to the hydrophobic surface increased with concentration. Substantially lower mass adsorbed was seen at the lowest protein concentration than was adsorbed at the next highest concentration. For PVC, very close to the same mass was adsorbed at the two higher concentrations, possibly saturating the binding area of the surface. These comparisons help characterize adsorption behavior at the hydrophobic polymer surface.

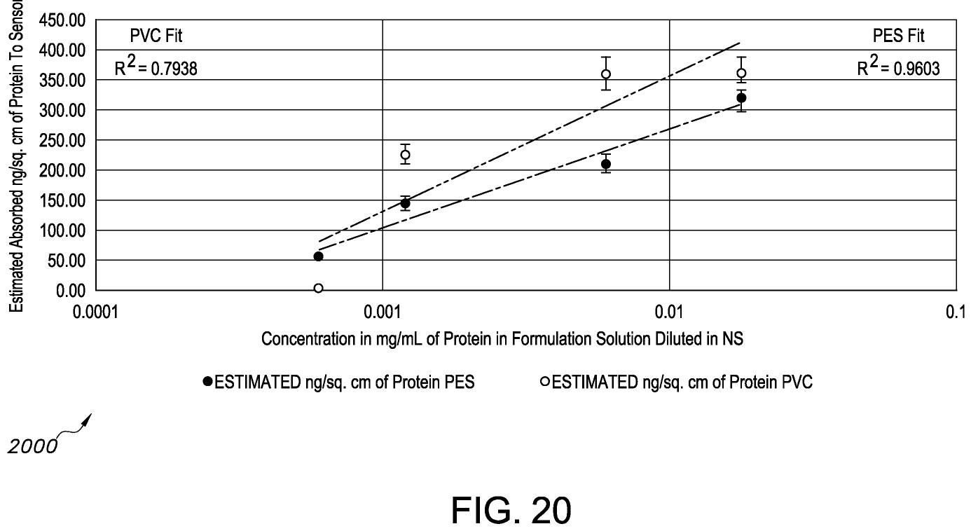

FIG. 20 is a diagram 2000 that illustrates mass contributions of the protein part of layer at PVC and PES sensor surface at different concentrations. This information indicated that there is a strong correlation between concentration in mg/mL of protein and estimated average mass of protein adsorbed at the polymer surface for both materials. Slightly lower amounts of protein were estimated to adsorb in the lowest concentration conditions, but in general the trend holds.

In FIG. 20, the relationship between concentration and adsorption is apparent, and this relates dose and concentration as further seen in FIGS. 21A-D. At higher concentrations of protein higher percent recovery results were seen, and higher amounts of adsorbed protein were also measured via QCM. As the dose increased, the amount of protein adsorbing also increased, but it did not increase to a degree enough to take up larger and larger percent of the total dose as concentration increased. Carrying through the 70% or greater limit often used as a benchmark in dose accuracy studies from FIGS. 21A and 21C to FIGS. 21B and 21D yielded a lowest useable concentration based on adsorption and content assays in these surfactant-poor environments of 0.0034 mg/ml for the filtered setup, and 0.00102 mg/mL for the unfiltered setup, which showed the effect inline filtration has on the protein (e.g., antibody therapeutic, etc.). All functions were either polynomials fitting exactly the data or lines of best fit which had an over 0.8 R2 value.

These results were based on calculations and estimates that use real world experimental data that is very basically processed. Correlating the data was pursued, and the relationships found however were very strong correlations between results of both experiments. A 1:1 correspondence of the ECLIA results to the adsorbed estimate masses found in QCM at the corresponding concentrations was not observed. This data primarily considered solely adsorption behavior of the protein drug and PS20 only using simple equations using a large amount of QCM experimental data, and queried its relationship concerning two conditions and polymers, to dose.

A final small number of experiments were done to verify whether the other excipients in the formulated solution diluted in NS were appreciably different than the normal saline solution. During the runs, the effect on frequency and by relation mass data was tested by running as a sample solution the formulation minus PS20 or protein, diluted to the same concentration as the corresponding surfactant and drug-containing solutions. This prior performed set of experiments confirmed the mass averages between the diluted formulation and the normal saline solution neat were not appreciably different and therefor the other components of the formulation besides PS and protein were not contributors to adsorbed masses.

Referring again to FIGS. 21A-D, filtered and unfiltered models of percent recovery and QCM Results are illustrated in which FIG. 21A is the QCM-predicted average amount adsorbed to the entire IV set with filtration versus the amount determined by content assay that was left on the IV set with filtration, and FIG. 21B is the QCM-predicted average amount adsorbed to the entire IV set with filtration versus the concentration of protein in the prepared DP. FIGS. 21C and 21D are analogous to FIGS. 21A and FIG. 21B data except this data is the without filtration set. Polynomials were fit to the experimental data, and unfiltered data was fit to three points instead of four due to inconclusive results at the highest concentration for percent recovery. Logarithmic functions of best fit were determined here for all data. The cutoff of 70% recovery is shown in FIGS. 21A and 21C and the corresponding QCM adsorbed amounts were inserted into the functions of FIGS. 21B and 21D to determine minimum useable concentrations for filtered and unfiltered conditions.

Protein 4 Experiments. In a further set of experiments in relation to protein 4, a higher molar ratio of approximately 281-2820 surfactant: protein was tested by varying the surfactant and holding the protein constant and low. Testing was conducted with various receptacle surface compositions such as those referenced above.

In particular, sample period masses were determined from triplicate runs of the same condition, and the conditions during the sample period were either: fully formulated DP with protein at a constant concentration of 0.00024 mg/mL with concentrations of PS20 of 0.000024%, 0.000048%, 0.00006%, or 0.00024% all in NS, fully formulated DP without protein with concentrations of PS20 at these four concentrations in NS, and fully formulated DP with protein at 0.00024 mg/mL without any PS20 in NS. The triplicate masses for the sample periods were averaged to form an average adsorbed mass for each condition. Sensors were used interchangeably and randomly after cleaning over all conditions and tested for reproducibility of the same results given the same conditions by multiple runs of similar conditions.

The equations 5 and 6 were used to estimate mass contributions at the polymer surface where x was the measured adsorbed mass in ng/cm2 when fully formulated IP diluted in NS without PS20 but with all other excipients and protein drug was sampled via QCM, y was the measured adsorbed mass in ng/cm2 when fully formulated investigational drug product (IP) diluted in NS with PS20 and all other excipients but no protein drug was sampled via QCM, and z was the measured adsorbed mass in ng/cm2 when fully formulated IP diluted in NS with PS20 and all other excipients and protein drug was sampled via QCM. This equation scheme was made for this specific case in that polysorbate mass and concentration is the focus of this study, the protein component of the masses was expected to be very small when compared to the polysorbate measurements, and overall the ratio of surfactant to protein was higher than previous experiments. This allowed for estimation of the comparatively small protein component and its relationship with the increasing concentrations of surfactant in the layer by simple mathematical comparison of each substance's propensity to contribute mass at the surface both individually, then together at once. These equations were more fit to understand formulation development when polysorbate was the varying quantity, instead of when both polysorbate and protein existed in a fixed ratio. The results of these QCM measurements and equations 5 and 6 were then compared to ECLIA content assay results.

Electrocheminuminescent Immunoassayed Infusion Experiment Methods. Infusion experiments were conducted in 250-mL NS IV bags with attached administration sets. The full IP formulation containing drug and PS20 at laddered concentrations as well as the other included excipients were diluted by admixture into the bag it was to be tested in in an ISO Class 5 Vertical Laminar Flow Hood using USP <797> aseptic techniques for sterile drug preparation. The bags were left at ambient room temperature and light for a 24-hour period, then infused into PETG bottles and samples were drawn up and diluted 1:10 in ECLIA assay buffer.

The results for percent recovery as measured with these experiments were then compared with the original solutions' concentrations. Unacceptable results via ECLIA were defined as ≥30% of dose difference from admixture of nominal concentration and the infusate collected in the PETG bottle. The diluent bags used for IP preparation were weighed before and after admixture as well as post-infusion. This allowed controlling for the specific fill volumes of each individual IV bag used and the exact concentration of IP preparation this corresponds to, which were very close to the nominal concentration levels at 0.0025 mg/mL thus making the PS20 concentrations between 0.000024% and 0.00024% also accurate. The same size IV bags of the size tested and the same type of IV line used in experiments were deconstructed and measured for internal fluid path surface area. Information for surface areas were then verified with manufacturers. The results of the percent recovery studies were compared with QCM results.

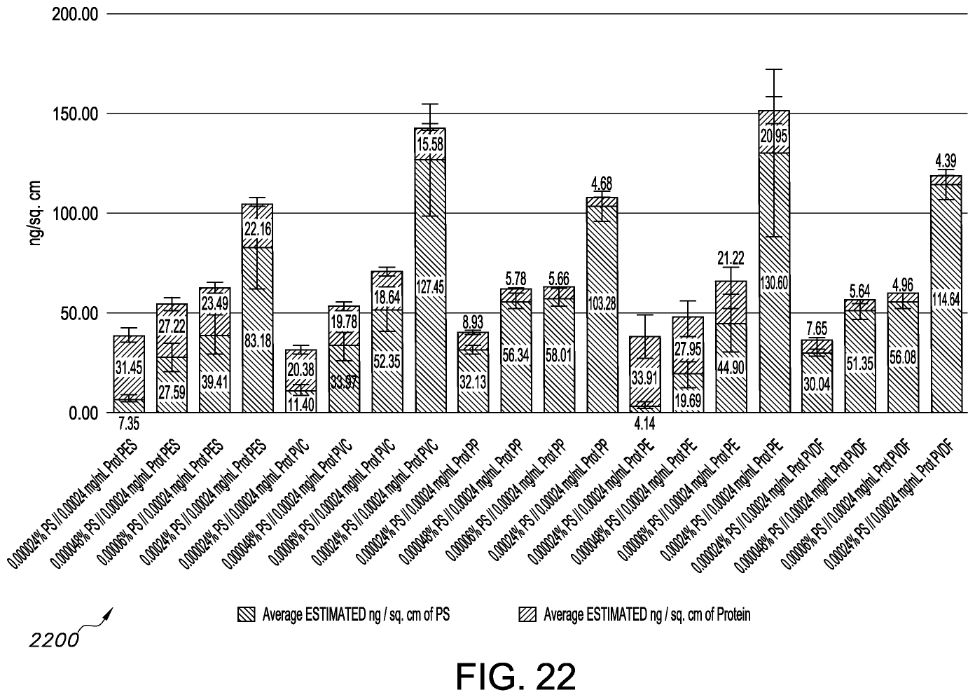

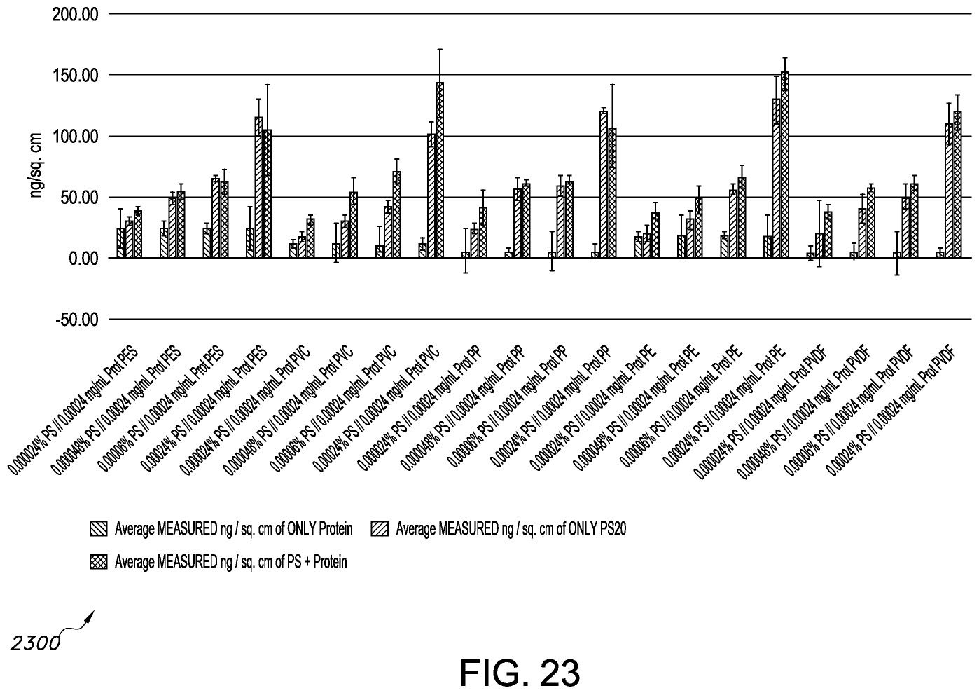

Average adsorbed amounts were measured for all three conditions and ECLIA infusion experiments were successful in measuring percent recovery for all conditions of fully formulated drug product. Adsorbed masses to all five polymer surfaces are shown in diagram 2200 of FIG. 22. The average contribution protein made to adsorbed layers by polymer were 34.52% [95% CI±10.46%] of the mass for PVC, 47.28% [95% CI±11.51%] for PES, 10.50% [95% CI±6.90%] for PVDF, 48.43% [95% CI±32.13%] for PE, and 11.07% [95% CI±7.32%] for PP but varied with polysorbate concentration. As polysorbate concentration went up, overall mass adsorbed increased, while protein component decreased, though protein concentration stayed constant. There was no significant difference between PVC, PP, and PE (IV set polymers), PES and PVDF (common filter polymers), or over all polymers in adsorbed mass when compared by ANOVA for corresponding concentrations and three conditions therein. In diagram 2300 of FIG. 23, in many cases the PS20 and protein average adsorbed masses were greater than polysorbate masses alone, save a few cases at the highest PS20 concentration. These protein component masses were then correlated with the percent PS20 concentrations. At highest PS20 concentration, the lowest amount of protein was adsorbed for all materials.

In particular, FIGS. 22 and 23 illustrate estimates of average mass contributions of PS20 and protein to layer at polymer sensor surface at different concentrations. In FIGS. 22 and 23 from left to right, adsorbed masses on PES, PVC, PP, PE, and PVDF at all four laddered concentrations of PS20. In FIG. 19, as PS20 concentration increased, protein component mass decreased. A tight range of the triplicate conditions tested are shown in FIG. 23, and these average condition masses constructed FIG. 22. In most cases PS20 made up most of the mass, except where PS20 is lower in concentration for a few conditions and polymers in FIG. 22.

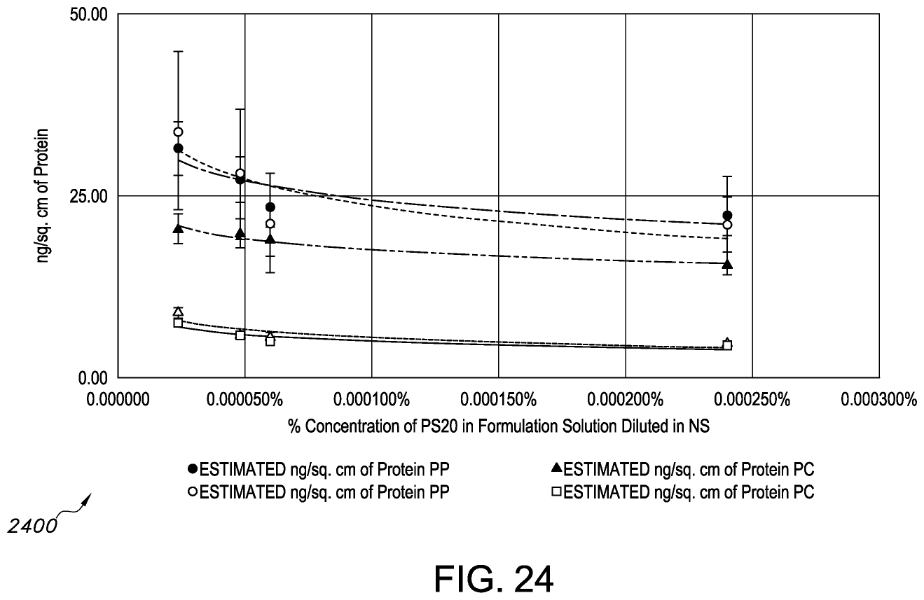

FIG. 24 is a diagram 2400 that illustrates estimates of protein average mass contributions of the protein part of layer at sensor surface at different PS20 concentrations. The relationship between concentration of PS20 surfactant and protein portion of mass is graphed here. As surfactant concentration increased, the protein adsorbed decreased. A log-linear function of best fit was plotted with R2 of all functions being greater than 0.9, and the error bars correspond to the 95% confidence interval around average adsorbed estimates.

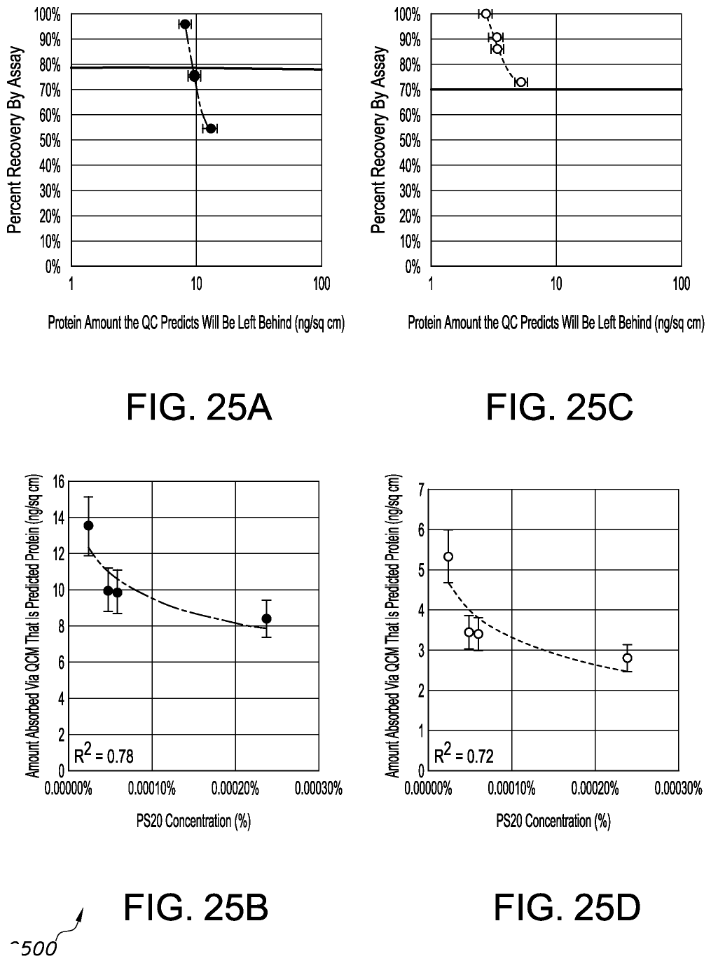

In diagram 2500 of FIGS. 25A-D, the percent recoveries for either filtered or unfiltered conditions were compared to the amounts of protein the QCM predicts being lost at increasing PS20 concentrations. Higher percent recoveries were associated with lower amounts of protein adsorbed, which was the opposite of what has been found in previous studies, and this was because of the different range of surfactant to protein ratio explored here. The PVC, PP, and PES QCM adsorption data for each material was used to model infusion experiments and compare to ECLIA-assayed results. The QCM data-modeled average adsorbed masses of protein to an IV set made of the same materials that had a defined fluid path the same as the infusion experiments had strong association with PS20 concentrations. This relationship and the polynomial relationship in FIGS. 25A and 25C allow finding the point where 70% or more of the dose is recovered post infusion in terms of PS20 concentration, which for the filtered condition was 0.000066% and for the unfiltered condition was 0.000013%.

Referring still to FIGS. 25A-D, these figures together illustrate filtered and unfiltered models of percent recovery and QCM results. FIG. 25A illustrates the QCM-predicted average amount adsorbed to the entire IV set with filtration versus the amount determined by content assay that was left on the IV set with filtration, and FIG. 25B illustrates the QCM-predicted average amount adsorbed to the entire IV set with filtration versus the concentration of PS20 in the prepared DP with constant protein concentration. FIGS. 25C and 25D are analogous to FIGS. 25A and 25B except this data is the without filtration set. Polynomials were fit to the experimental data in FIGS. 25A and 25C and finding the point where the polynomial crosses 70% for the DP under specific conditions dictates what concentration of PS20 in the clinical setting when using or not using a filter is allowable to preserve dose accuracy.

To try and account for possibly confounding effects of diluent as an ionic liquid altering frequency as a bulk liquid during the sample period or for other excipients in the formulation to affect average QCM-adsorbed mass measurements, a few other experiments and calculations were performed. Primarily, the diluent's effect was accounted for by running an diluent blank period before each sample period and subtracting off its effect as a bulk liquid from the sample signal seen. Also, a small number of experiments were done to verify whether the other excipients in the formulated solution diluted in diluent were appreciably different than the normal saline solution. This priorly performed set of experiments confirmed the mass averages between the diluted formulation of the same components and the normal saline solution neat were not appreciably different and therefore the other components of the formulation besides PS and protein were not contributors to adsorbed masses.

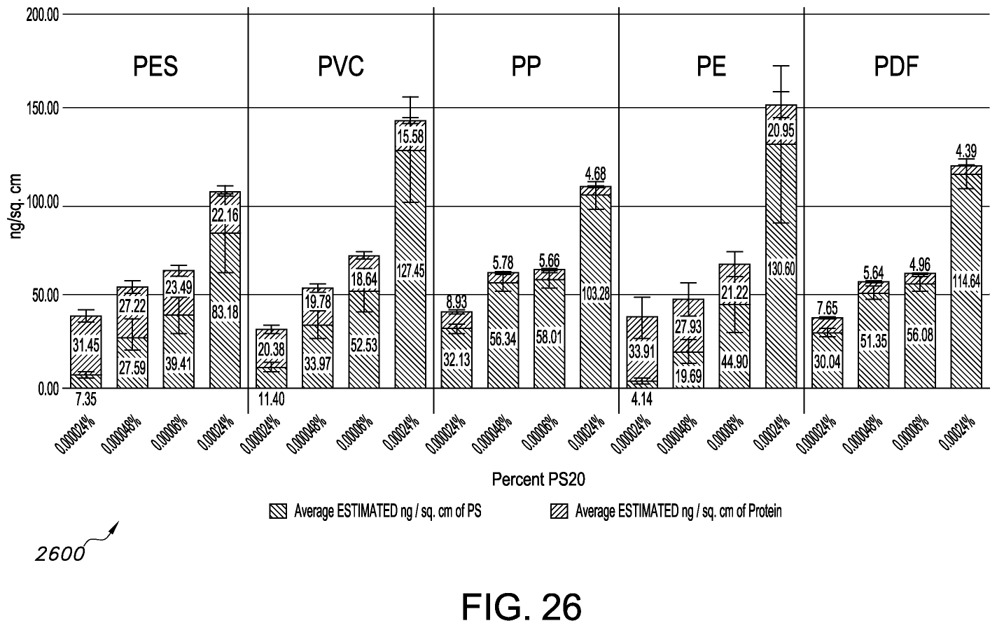

FIG. 26 is a diagram illustrating estimates of mass contributions of PS20 and protein 4 to layer at sensor surface at different concentrations. This diagram shows that the amount of protein lost as protein was kept constant as PS concentration goes up.

### EMBODIMENTS

The current subject matter includes the following non-limiting embodiments.

In one set of embodiments, provided are:

A1. A computer-implemented method comprising:
receiving data identifying a medication comprising a concentration of a drug product in a background fluid and a composition of a surface of a receptacle for housing the medication;
predicting, by a drug substance adsorption behavior model using the received data, a percent of dose lost and an interaction behavior between the medication and the receptacle; and
providing data characterizing the predicted percent of dose lost and the interaction behavior;
   wherein the drug substance adsorption behavior model is generated by:
   conducting a plurality of test measurements simulating delivery of the medication at various concentrations housed within receptacles having varying sizes and surface compositions;
   measuring, during each test measurement, acoustic resonances of at least one quartz crystal microbalance (QCM) sensor having a coating corresponding to the surface composition of the respective receptacle, wherein different frequencies of measured harmonics forming part of the acoustic resonances correlate to adsorbed drug product by the surface composition;
   determining, for each test measurement based on the measured acoustic resonances, a percent of dose lost and an interaction behavior between the medication and the receptacle; and
constructing the drug substance adsorption behavior model based on the determined percent of dose lost and the interaction behavior between the respective medications and the corresponding receptacles.

A2. The method of embodiment A1 further comprising generating the drug absorption behavior model.

A3. The method of embodiment A1 or A2, wherein the interaction behavior between the surface of the receptacle and the medication comprises how much of a surfactant or other component of the drug solution is adsorbed by the surface of the receptacle.

A4. The method of any of embodiments A1 to A3, wherein the predicted percent of dose lost is based on a period of time.

A5. The method of any of embodiments A1 to A4, wherein the predicted percent of dose lost is based on an amount of dose lost during administration of the medication.

A6. The method of any of embodiments A1 to A5, wherein the predicted percent of dose lost is based on an amount of dose lost during manufacture or preparation of the medication.

A7. The method of any of embodiments A1 to A6, wherein the predicted percent of dose lost is based on an amount of dose lost during storage of the medication.

A8. The method of any of embodiments A1 to A7, wherein the predicted percent of dose lost is based on an amount of dose lost during transportation of the medication.

A9. The method of any of embodiments A1 to A8, wherein the received data comprises a total possible medication contact surface area for the receptacle.

A10. The method of any of embodiments A1 to A9, wherein the receptacle comprises an intravenous fluid (IV) bag, IV line, a syringe, a pre-filled syringe, an inline filter, a needle, a catheter, intravenous tubing, or a vial.

A11. The method of any of embodiments A1 to A10, wherein the surface comprises at least one surface involved in manufacture, storage, administration, preparation, or transportation of the drug product.

A12. The method of any of embodiments A1 to A11, wherein the surface is selected from a group consisting of: polyvinyl chloride (PVC), polypropylene (PP), polyvinylidene fluoride (PVDF), polyvinyl chloride (PV), polyethersulfone (PES), polyethylene (PE), polycarbonate (PC), polyurethane (PUR), nylon, boro-silicate glass, and steel.

A13. The method of any of embodiments A1 to A11, wherein the surface is selected from a group consisting of: basic elements, oxides, nitrides, carbides, sulfides, polymers, functionalized molecules, glasses, steels, and alloys.

A14. The method of any of embodiments A1 to A13, wherein background fluid is selected from a group consisting of: normal saline (NS), half-normal saline, 3% normal saline, lactated Ringer's solution, plasmalyte, dextrose 5% in water, dextrose 5% in water and half-normal saline, dextrose 5% and lactated Ringer's solution, 7.5% sodium bicarbonate, albumin 5%, albumin 25%, 10% dextran 40 in NS, hetastarch 6% in NS, normosol-r, normosol-m, and hypertonic saline.

A15. The method of any of embodiments A1 to A14, wherein providing data characterizing the predicted percent of dose lost and the interaction behavior between the receptacle and the medication comprises: causing the data to be displayed in electronic visual display, transmitting the data over a computing network to a remote computing system, loading the data into memory, or storing the data in physical persistence.

A16. The method of any of embodiments A1 to A15, wherein the drug product comprises a protein, a nucleic acid, a lipid or a virus that is adsorbed by the surface of the receptacle.

A17. The method of any of embodiments A1 to A16, wherein the protein comprises an antibody, an antibody-drug conjugate, or a fusion protein that contacts the surface of the receptacle that contacts the surface of the receptacle.

A18. The method of any of embodiments A1 to A17, wherein the drug substance adsorbance behavior model is further generated by:
estimating a contribution of mass of protein at the surface equal to z (1−x/y);
wherein:
   x is a measured adsorbed mass of the medication in a first state;
   y is a measured adsorbed mass of the medication in a second state; and
   z is a measured adsorbed mass of the medication in a third state.

A19. The method of any of embodiments A1 to A18, wherein the drug substance adsorbance behavior model is further generated by:
estimating a contribution of mass of a surfactant at the surface equal to z*(x/y).
   wherein:
x is a measured adsorbed mass of the medication in a first state;
y is a measured adsorbed mass of the medication in a second state; and
z is a measured adsorbed mass of the medication in a third state.

A20. The method of any of embodiments A1 to A17, wherein the drug substance adsorbance behavior model is further generated by:
estimating a contribution of mass of protein at the surface equal to z (1−y/x);
wherein:
   x is a measured adsorbed mass of the medication in a first state;
   y is a measured adsorbed mass of the medication in a second state; and
   z is a measured adsorbed mass of the medication in a third state.

A21. The method of any of embodiments A1 to A17 and A20, wherein the drug substance adsorbance behavior model is further generated by:
estimating a contribution of mass of a surfactant at the surface equal to z*(x/y);
wherein:
   x is a measured adsorbed mass of the medication in a first state;
   y is a measured adsorbed mass of the medication in a second state; and
   z is a measured adsorbed mass of the medication in a third state.

A22. The method of any of embodiments A1 to A17 wherein:
when a molar ratio of surfactant to protein is below a pre-defined value, the drug substance adsorbance behavior model is generated by:
   estimating a contribution of mass of protein at the surface equal to z (1−x/y); and
      estimating a contribution of mass of a surfactant at the surface equal to z*(x/y);
when a molar ratio of surfactant to protein is equal to or above a pre-defined value, the drug substance adsorbance behavior model is generated by:
   estimating a contribution of mass of protein at the surface equal to z (1−y/x); and
   estimating a contribution of mass of a surfactant at the surface equal to z * (x/y);
x is a measured adsorbed mass of the medication in a first state;
y is a measured adsorbed mass of the medication in a second state; and
z is a measured adsorbed mass of the medication in a third state.

A23. A computer-implemented method for screening polymers for medication receptacles comprising:
receiving data identifying a medication comprising a concentration of a drug product in a background fluid and a polymeric composition of a surface of a receptacle for housing the medication;
predicting, by a drug substance adsorption behavior model using the received data, a percent of dose lost and an interaction behavior between the medication and the receptacle, the drug substance absorption behavior model being generated using one or more empirical tests using quartz crystal microbalance sensors; and
providing data characterizing the predicted percent of dose lost and the interaction behavior.

A24. The method as in any embodiments A1 to A23 further comprising:
loading a medical receptacle with the medication based on at least one of the predicted percent of dose lost or the interaction behavior.

In another set of embodiments, provided are:

B1. A system comprising:
at least one data processor; and
memory storing instructions which, when executed by the at least one data processor, implement operations comprising:
   receiving data identifying a medication comprising a concentration of a drug product in a background fluid and a composition of a surface of a receptacle for housing the medication;
   predicting, by a drug substance adsorption behavior model using the received data, a percent of dose lost and an interaction behavior between the medication and the receptacle; and
   providing data characterizing the predicted percent of dose lost and the interaction behavior;
wherein the drug substance adsorption behavior model is generated by:
   conducting a plurality of test measurements simulating delivery of the medication at various concentrations housed within receptacles having varying sizes and surface compositions;
   measuring, during each test measurement, acoustic resonances of at least one quartz crystal microbalance (QCM) sensor having a coating corresponding to the surface composition of the respective receptacle, wherein different frequencies of measured harmonics forming part of the acoustic resonances correlate to adsorbed drug product by the surface composition;
   determining, for each test measurement based on the measured acoustic resonances, a percent of dose lost and an interaction behavior between the medication and the receptacle; and
   constructing the drug substance adsorption behavior model based on the determined percent of dose lost and the interaction behavior between the respective medications and the corresponding receptacles.

B2. The system of embodiment B1, wherein the operations further comprise: generating the drug absorption behavior model.

B3. The system of embodiment B1 or B2, wherein the interaction behavior between the surface of the receptacle and the medication comprises how much of a surfactant or other component of the drug solution is adsorbed by the surface of the receptacle.

B4. The system of any of embodiments B1 to B3, wherein the predicted percent of dose lost is based on a period of time.

B5. The system of any of embodiments B1 to B4, wherein the predicted percent of dose lost is based on an amount of dose lost during administration of the medication.

B6. The system of any of embodiments B1 to B5, wherein the predicted percent of dose lost is based on an amount of dose lost during manufacture or preparation of the medication.

B7. The system of any of embodiments B1 to B6, wherein the predicted percent of dose lost is based on an amount of dose lost during storage of the medication.

B8. The system of any of embodiments B1 to B7, wherein the predicted percent of dose lost is based on an amount of dose lost during transportation of the medication.

B9. The system of any of embodiments B1 to B8, wherein the received data comprises a total possible medication contact surface area for the receptacle.

B10. The system of any of embodiments B1 to B9, wherein the receptacle comprises an intravenous fluid (IV) bag, IV line, a syringe, a pre-filled syringe, an inline filter, a needle, a catheter, intravenous tubing, or a vial.

B11. The system of any of embodiments B1 to B10, wherein the surface comprises at least one surface involved in manufacture, storage, administration, preparation, or transportation of the drug product.

B12. The system of any of embodiments B1 to B11, wherein the surface is selected from a group consisting of: polyvinyl chloride (PVC), polypropylene (PP), polyvinylidene flouride (PVDF), polyvinyl chloride (PV), polyethersulfone (PES), polyethylene (PE), polycarbonate (PC), polyurethane (PUR), nylon, boro-silicate glass, and steel.

B13. The system of any of embodiments B1 to B11, wherein the surface is selected from a group consisting of: basic elements, oxides, nitrides, carbides, sulfides, polymers, functionalized molecules, glasses, steels, and alloys.

B14. The system of any of embodiments B1 to B13, wherein background fluid is selected from a group consisting of: normal saline (NS), half-normal saline, 3% normal saline, lactated Ringer's solution, plasmalyte, dextrose 5% in water, dextrose 5% in water and half-normal saline, dextrose 5% and lactated Ringer's solution, 7.5% sodium bicarbonate, albumin 5%, albumin 25%, 10% dextran 40 in NS, hetastarch 6% in NS, normosol-r, normosol-m., and hypertonic saline.

B15. The system of any of embodiments B1 to B14, wherein providing data characterizing the predicted percent of dose lost and the interaction behavior between the receptacle and the medication comprises: causing the data to be displayed in electronic visual display, transmitting the data over a computing network to a remote computing system, loading the data into memory, or storing the data in physical persistence.

B16. The method of any of embodiments B1 to B15, wherein the drug product comprises a protein, a nucleic acid, a lipid or a virus that is adsorbed by the surface of the receptacle.

B17. The method of any of embodiments A1 to A16, wherein the protein comprises an antibody, an antibody-drug conjugate, or a fusion protein that contacts the surface of the receptacle that contacts the surface of the receptacle.

B18. The system of any of embodiments B1 to B17, wherein the drug substance adsorbance behavior model is further generated by:
estimating a contribution of mass of protein at the surface equal to z (1−x/y);
   wherein:
      x is a measured adsorbed mass of the medication in a first state;
      y is a measured adsorbed mass of the medication in a second state; and
      z is a measured adsorbed mass of the medication in a third state.

B19. The system of any of embodiments B1 to B18, wherein the drug substance adsorbance behavior model is further generated by: estimating a contribution of mass of a surfactant at the surface equal to z*(x/y);
wherein:
   x is a measured adsorbed mass of the medication in a first state;
   y is a measured adsorbed mass of the medication in a second state; and
   z is a measured adsorbed mass of the medication in a third state.

B20. The system of any of embodiments B1 to B17, wherein the drug substance adsorbance behavior model is further generated by:
estimating a contribution of mass of protein at the surface equal to z (1−y/x);
wherein:
   x is a measured adsorbed mass of the medication in a first state;
   y is a measured adsorbed mass of the medication in a second state; an
   z is a measured adsorbed mass of the medication in a third state.

B21. The system of any of embodiments B1 to B17 and B20, wherein the drug substance adsorbance behavior model is further generated by:
estimating a contribution of mass of a surfactant at the surface equal to z*(x/y);
wherein:
   x is a measured adsorbed mass of the medication in a first state;
   y is a measured adsorbed mass of the medication in a second state; and
   z is a measured adsorbed mass of the medication in a third state.

B22. The system of any of embodiments B1 to B17 wherein:
when a molar ratio of surfactant to protein is below a pre-defined value, the drug substance adsorbance behavior model is generated by:
   estimating a contribution of mass of protein at the surface equal to z (1−x/y); and
      estimating a contribution of mass of a surfactant at the surface equal to z*(x/y);
when a molar ratio of surfactant to protein is equal to or above a pre-defined value, the drug substance adsorbance behavior model is generated by:
   estimating a contribution of mass of protein at the surface equal to z (1−y/x); and
   estimating a contribution of mass of a surfactant at the surface equal to z * (x/y);
x is a measured adsorbed mass of the medication in a first state;
y is a measured adsorbed mass of the medication in a second state; and
z is a measured adsorbed mass of the medication in a third state.

B23. A system for screening polymers for medication receptacles comprising:
at least one data processor; and
memory storing instructions which, when executed by the at least one data processor, result in operations comprising:
   receiving data identifying a medication comprising a concentration of a drug product in a background fluid and a polymeric composition of a surface of a receptacle for housing the medication;
   predicting, by a drug substance adsorption behavior model using the received data, a percent of dose lost and an interaction behavior between the medication and the receptacle, the drug substance absorption behavior model being generated using one or more empirical tests using quartz crystal microbalance sensors; and
   providing data characterizing the predicted percent of dose lost and the interaction behavior.

B24. An apparatus comprising:
means for receiving data identifying a medication comprising a concentration of a drug product in a background fluid and a polymeric composition of a surface of a receptacle for housing the medication;
means predicting, by a drug substance adsorption behavior model using the received data, a percent of dose lost and an interaction behavior between the medication and the receptacle, the drug substance absorption behavior model being generated using one or more empirical tests using quartz crystal microbalance sensors; and
means for providing data characterizing the predicted percent of dose lost and the interaction behavior.

In another set of embodiments, provided are:

C1. A computer-implemented method comprising:
conducting a plurality of test measurements simulating delivery of medication at various concentrations housed within receptacles having varying sizes and surface compositions;
measuring, during each test measurement, acoustic resonances of at least one quartz crystal microbalance (QCM) sensor having a coating corresponding to a surface composition of the respective receptacle, wherein different frequencies of measured harmonics forming part of the acoustic resonances correlate to adsorbed drug product by the surface composition;
determining, for each test measurement based on the measured acoustic resonances, a percent of dose lost and an interaction behavior between the medication and the receptacle; and
constructing a drug substance adsorption behavior model based on the determined percent of dose lost and the interaction behavior between the respective medications and the corresponding receptacles.

C2. The method of embodiment C1 further comprising:
receiving data identifying a medication comprising a concentration of a drug product in a background fluid and a composition of a surface of a receptacle for housing the medication;
predicting, by the drug substance adsorption behavior model using the received data, a percent of dose lost and an interaction behavior between the medication and the receptacle; and
providing data characterizing the predicted percent of dose lost and the interaction behavior.

C3. The method of embodiment C2, wherein the interaction behavior between the surface of the receptacle and the medication comprises how much of a surfactant or other component of the drug solution is adsorbed by the surface of the receptacle.

C4. The method of embodiment C2 or C3, wherein the predicted percent of dose lost is based on a period of time.

C5. The method of any of embodiments C2 to C4, wherein the predicted percent of dose lost is based on an amount of dose lost during administration of the medication.

C6. The method of any of embodiments C2 to C5, wherein the predicted percent of dose lost is based on an amount of dose lost during manufacture or preparation of the medication.

C7. The method of any of embodiments C2 to C6, wherein the predicted percent of dose lost is based on an amount of dose lost during storage of the medication.

C8. The method of any of embodiments C2 to C7, wherein the predicted percent of dose lost is based on an amount of dose lost during transportation of the medication.

C9. The method of any of embodiments C2 to C8, wherein the received data comprises a total possible medication contact surface area for the receptacle.

C10. The method of any of embodiments C2 to C9, wherein the receptacle comprises an intravenous fluid (IV) bag, IV line, a syringe, a pre-filled syringe, an inline filter, a needle, a catheter, intravenous tubing, or a vial.

C11. The method of any of embodiments C2 to C10, wherein the surface comprises at least one surface involved in manufacture, storage, administration, preparation, or transportation of the drug product.

C12. The method of any of embodiments C2 to C11, wherein the surface is selected from a group consisting of: polyvinyl chloride (PVC), polypropylene (PP), polyvinylidene flouride (PVDC), polyvinyl chloride (PV), polyethersulfone (PES), polyethylene (PE), polycarbonate (PC), polyurethane (PUR), nylon, boro-silicate glass, and steel.

C13. The method of any of embodiments C2 to C11, wherein the surface is selected from a group consisting of: basic elements, oxides, nitrides, carbides, sulfides, polymers, functionalized molecules, glasses, steels, and alloys

C14. The method of any of embodiments C2 to C13, wherein background fluid is selected from a group consisting of: normal saline (NS), half-normal saline, 3% normal saline, lactated Ringer's solution, plasmalyte, dextrose 5% in water, dextrose 5% in water and half-normal saline, dextrose 5% and lactated Ringer's solution, 7.5% sodium bicarbonate, albumin 5%, albumin 25%, 10% dextran 40 in NS, hetastarch 6% in NS, normosol-r, normosol-m., and hypertonic saline.

C15. The method of any of embodiments C2 to C14, wherein providing data characterizing the predicted percent of dose lost and the interaction behavior between the receptacle and the medication comprises: causing the data to be displayed in electronic visual display, transmitting the data over a computing network to a remote computing system, loading the data into memory, or storing the data in physical persistence.

C16. The method of any of embodiments C2 to C15, wherein the drug product comprises a monoclonal antibody, an antibody-drug conjugate, proteins, or cells that is adsorbed by the surface of the receptacle.

C17. The method of any of embodiments C2 to C16, wherein the drug product comprises nucleic acid, cells, viruses, or lipids that contact the surface of the receptacle.

C18. The method of any of the preceding embodiments, wherein the drug substance adsorbance behavior model is further generated by:
estimating a contribution of mass of protein at the surface equal to z (1−X/Y);
   wherein:
      x is a measured adsorbed mass of the medication in a first state;
      y is a measured adsorbed mass of the medication in a second state; and
      z is a measured adsorbed mass of the medication in a third state.

C19. The method of any of the preceding embodiments, wherein the drug substance adsorbance behavior model is further generated by:
estimating a contribution of mass of a surfactant at the surface equal to z*(x/y);
   wherein:
      x is a measured adsorbed mass of the medication in a first state;
      y is a measured adsorbed mass of the medication in a second state; and
      z is a measured adsorbed mass of the medication in a third state.

C20. The method of any of embodiments C1 to C17, wherein the drug substance adsorbance behavior model is further generated by:
estimating a contribution of mass of protein at the surface equal to z (1−y/x);
wherein:
   x is a measured adsorbed mass of the medication in a first state;
   y is a measured adsorbed mass of the medication in a second state; an
   z is a measured adsorbed mass of the medication in a third state.

C21. The method of any of embodiments C1 to C17 and C20, wherein the drug substance adsorbance behavior model is further generated by:
estimating a contribution of mass of a surfactant at the surface equal to z*(x/y);
wherein:
   x is a measured adsorbed mass of the medication in a first state;
   y is a measured adsorbed mass of the medication in a second state; and
   z is a measured adsorbed mass of the medication in a third state.

C22. The method of any of embodiments C1 to C17 wherein:
when a molar ratio of surfactant to protein is below a pre-defined value, the drug substance adsorbance behavior model is generated by:
   estimating a contribution of mass of protein at the surface equal to z (1−x/y); and
      estimating a contribution of mass of a surfactant at the surface equal to z*(x/y);
when a molar ratio of surfactant to protein is equal to or above a pre-defined value, the drug substance adsorbance behavior model is generated by:
   estimating a contribution of mass of protein at the surface equal to z (1−y/x); and
   estimating a contribution of mass of a surfactant at the surface equal to z * (x/y);
x is a measured adsorbed mass of the medication in a first state;
y is a measured adsorbed mass of the medication in a second state; and
z is a measured adsorbed mass of the medication in a third state.

C23. The method as in any embodiments C1 to C22 further comprising:
loading a medical receptacle with the medication based on values generated by the constructed drug substance adsorption behavior model.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. For example, the current subject matter is applicable to a wide variety of surfactants, materials, diluents and the like and should not, unless otherwise specified, be limited to the examples provided herein. Instead, they are merely some examples consistent with aspects related to the described subject matter. Although a few variations have been described in detail above, other modifications or additions are possible. Further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the order shown, or sequential order, to achieve desirable results. Other implementations may be within the scope of the following claims.

1. A computer-implemented method comprising:
receiving data identifying a medication comprising a concentration of a drug product in a background fluid and a composition of a surface of a receptacle for housing the medication;
predicting, by a drug substance adsorption behavior model using the received data, a percent of dose lost and an interaction behavior between the medication and the receptacle; and
providing data characterizing the predicted percent of dose lost and the interaction behavior;
wherein the drug substance adsorption behavior model is generated by:
conducting a plurality of test measurements simulating delivery of the medication at various concentrations housed within receptacles having varying sizes and surface compositions;
measuring, during each test measurement, acoustic resonances of at least one quartz crystal microbalance (QCM) sensor having a coating corresponding to the surface composition of the respective receptacle, wherein different frequencies of measured harmonics forming part of the acoustic resonances correlate to adsorbed drug product by the surface composition;
determining, for each test measurement based on the measured acoustic resonances, a percent of dose lost and an interaction behavior between the medication and the receptacle; and
constructing the drug substance adsorption behavior model based on the determined percent of dose lost and the interaction behavior between the respective medications and the corresponding receptacles.

2. The method of claim 1 further comprising generating the drug absorption behavior model.

3. The method of claim 1, wherein the interaction behavior between the surface of the receptacle and the medication comprises how much of a surfactant or other component of the drug solution is adsorbed by the surface of the receptacle.

4. The method of claim 1, wherein the predicted percent of dose lost is based on a period of time.

5. The method of claim 1, wherein the predicted percent of dose lost is based on an amount of dose lost during administration of the medication.

6. The method of claim 1, wherein the predicted percent of dose lost is based on an amount of dose lost during manufacture or preparation of the medication.

7. The method of claim 1, wherein the predicted percent of dose lost is based on an amount of dose lost during storage of the medication.

8. The method of claim 1, wherein the predicted percent of dose lost is based on an amount of dose lost during transportation of the medication.

9. The method of claim 1, wherein the received data comprises a total possible medication contact surface area for the receptacle.

10. The method of claim 1, wherein the receptacle comprises an intravenous fluid (IV) bag, IV line, a syringe, a pre-filled syringe, an inline filter, a needle, a catheter, intravenous tubing, or a vial.

11. The method of claim 1, wherein the surface comprises at least one surface involved in manufacture, storage, administration, preparation, or transportation of the drug product.

12. The method of claim 1, wherein the surface is selected from a group consisting of: polyvinyl chloride (PVC), polypropylene (PP), polyvinylidene flouride (PVDF), polyvinyl chloride (PV), polyethersulfone (PES), polyethylene (PE), polycarbonate (PC), polyurethane (PUR), nylon, boro-silicate glass, and steel.

13. The method of claim 1, wherein the surface is selected from a group consisting of: basic elements, oxides, nitrides, carbides, sulfides, polymers, functionalized molecules, glasses, steels, and alloys.

14. The method of claim 1, wherein background fluid is selected from a group consisting of: normal saline (NS), half-normal saline, 3% normal saline, lactated Ringer's solution, plasmalyte, dextrose 5% in water, dextrose 5% in water and half-normal saline, dextrose 5% and lactated Ringer's solution, 7.5% sodium bicarbonate, albumin 5%, albumin 25%, 10% dextran 40 in NS, hetastarch 6% in NS, normosol-r, normosol-m., and hypertonic saline.

15. The method of claim 1, wherein providing data characterizing the predicted percent of dose lost and the interaction behavior between the receptacle and the medication comprises: causing the data to be displayed in electronic visual display, transmitting the data over a computing network to a remote computing system, loading the data into memory, or storing the data in physical persistence.

16. The method of claim 1, wherein the drug product comprises a protein, a nucleic acid, a lipid or a virus that is adsorbed by the surface of the receptacle.

17. The method of claim 16, wherein the protein comprises an antibody, an antibody-drug conjugate, or a fusion protein that contacts the surface of the receptacle.

18. The method of claim 1, wherein the drug substance adsorbance behavior model is further generated by:
estimating a contribution of mass of protein at the surface equal to z (1−x/y);
wherein:
x is a measured adsorbed mass of the medication in a first state;
y is a measured adsorbed mass of the medication in a second state; and
z is a measured adsorbed mass of the medication in a third state.

19. The method of claim 1, wherein the drug substance adsorbance behavior model is further generated by:
estimating a contribution of mass of a surfactant at the surface equal to z*(x/y);
wherein:
x is a measured adsorbed mass of the medication in a first state;
y is a measured adsorbed mass of the medication in a second state; and
z is a measured adsorbed mass of the medication in a third state.

20. The method of claim 1, wherein the drug substance adsorbance behavior model is further generated by:
estimating a contribution of mass of protein at the surface equal to z (1−y/x);
wherein:
x is a measured adsorbed mass of the medication in a first state;
y is a measured adsorbed mass of the medication in a second state; and
z is a measured adsorbed mass of the medication in a third state.

21. The method of claim 1, wherein the drug substance adsorbance behavior model is further generated by:
estimating a contribution of mass of a surfactant at the surface equal to z*(x/y);
wherein:
x is a measured adsorbed mass of the medication in a first state;
y is a measured adsorbed mass of the medication in a second state; and
z is a measured adsorbed mass of the medication in a third state.

22. The method of claim 1, wherein:
when a molar ratio of surfactant to protein is below a pre-defined value, the drug substance adsorbance behavior model is generated by:
estimating a contribution of mass of protein at the surface equal to z (1−x/y); and
estimating a contribution of mass of a surfactant at the surface equal to z*(x/y);
when a molar ratio of surfactant to protein is equal to or above a pre-defined value, the drug substance adsorbance behavior model is generated by:
estimating a contribution of mass of protein at the surface equal to z (1−y/x); and
estimating a contribution of mass of a surfactant at the surface equal to z*(x/y);
x is a measured adsorbed mass of the medication in a first state;
y is a measured adsorbed mass of the medication in a second state; and
z is a measured adsorbed mass of the medication in a third state.

23. A computer-implemented method for screening polymers for medication receptacles comprising:
receiving data identifying a medication comprising a concentration of a drug product in a background fluid and a polymeric composition of a surface of a receptacle for housing the medication;
predicting, by a drug substance adsorption behavior model using the received data, a percent of dose lost and an interaction behavior between the medication and the receptacle, the drug substance absorption behavior model being generated using one or more empirical tests using quartz crystal microbalance sensors; and
providing data characterizing the predicted percent of dose lost and the interaction behavior.

24. A method of claim 1, further comprising:
loading a medical receptacle with the medication based on at least one of the predicted percent of dose lost or the interaction behavior.

25. A system comprising:
at least one data processor; and
memory storing instructions which, when executed by the at least one data processor, implement a method of claim 1.

26. An apparatus comprising:
means for receiving data identifying a medication comprising a concentration of a drug product in a background fluid and a polymeric composition of a surface of a receptacle for housing the medication;
means predicting, by a drug substance adsorption behavior model using the received data, a percent of dose lost and an interaction behavior between the medication and the receptacle, the drug substance absorption behavior model being generated using one or more empirical tests using quantum crystal microbalance sensors; and
means for providing data characterizing the predicted percent of dose lost and the interaction behavior.

27. A computer-implemented method comprising:
conducting a plurality of test measurements simulating delivery of medication at various concentrations housed within receptacles having varying sizes and surface compositions;
measuring, during each test measurement, acoustic resonances of at least one quartz crystal microbalance (QCM) sensor having a coating corresponding to a surface composition of the respective receptacle, wherein different frequencies of measured harmonics forming part of the acoustic resonances correlate to adsorbed drug product by the surface composition;
determining, for each test measurement based on the measured acoustic resonances, a percent of dose lost and an interaction behavior between the medication and the receptacle; and
constructing a drug substance adsorption behavior model based on the determined percent of dose lost and the interaction behavior between the respective medications and the corresponding receptacles.

28. The method of claim 27 further comprising:
receiving data identifying a medication comprising a concentration of a drug product in a background fluid and a composition of a surface of a receptacle for housing the medication;
predicting, by the drug substance adsorption behavior model using the received data, a percent of dose lost and an interaction behavior between the medication and the receptacle; and
providing data characterizing the predicted percent of dose lost and the interaction behavior.
